(19) United States
(12) Patent Application Publication
(10) Pub. No.: US 2022/0081714 A1
(43) Pub. Date: Mar. 17, 2022

(54) STORING TEMPORAL DATA INTO DNA

(71) Applicants: Northwestern University, Evanston, IL (US); The Trustees of the University of Pennsylvania, Philadelphia, PA (US); The Trustees of Columbia University in the City of New York, New York, NY (US)

(72) Inventors: Keith E.J. Tyo, Evanston, IL (US); Namita Bhan, Evanston, IL (US); Konrad Kording, Philadelphia, PA (US); Joshua Glaser, New York, NY (US); Johathan Strutz, Evanston, IL (US); Alec Castinado, Chicago, IL (US)

(73) Assignees: Northwestern University, Evanston, IL (US); The Trustees of the University of Pennsylvania, Philadelphia, PA (US); The Trustees of Columbia University in the City of New York, New York, NY (US)

(21) Appl. No.: 17/420,606

(22) PCT Filed: Jan. 6, 2020
(86) PCT No.: PCT/US2020/012358

§ 371 Date: Jul. 2, 2021

Related U.S. Application Data
(60) Provisional application No. 62/788,614, filed on Jan. 4, 2019.

(51) Int. Cl. C12Q 1/6869

(52) U.S. Cl. CPC C12Q 1/6869

(57) ABSTRACT

Provided herein are systems and methods for using DNA polymerases to record information onto DNA for single cell high time-resolution recording and for high density data storage. The technology provides a DNA polymerase-based nano scale device that can be genetically encoded to record temporal information about the polymerase's environment into an extending single stand of DNA.

### RELATED APPLICATION

This application claims priority under 35 U.S.C. § 119(e) to U.S. Provisional Application 62/788,614 filed Jan. 4, 2019, the entire contents of which are incorporated herein by reference.

### STATEMENT REGARDING FEDERAL FUNDING

This invention was made with government support under MH103910 and NS107697 awarded by the National Institutes of Health. The government has certain rights in the invention

### FIELD

Provided herein are systems and methods for using DNA polymerases to record information onto DNA for single cell high time-resolution recording and for high density data storage.

### BACKGROUND

The following discussion is provided to aid the reader in understanding the disclosure and is not admitted to describe or constitute prior art thereto.

Measuring bio-signals that span a large range of spatial and temporal scales is critical to understanding complex biological phenomena. In many systems, analytical techniques must probe many cells simultaneously to capture system-level effects, including cells deep in a tissue without disturbing the biological environment. A particularly challenging problem is the measurement of molecules at cellular (or subcellular) spatial resolution and sub-minute temporal resolution in crowded environments. For example, in neuroscience, it is desirable to record neural firing over time across many neurons in brain tissue. Many other recording scenarios are also complex systems, such as in developmental biology and microbial biofilms, where dynamic waves of signaling molecules determine function. Thus there is a need to study time-dependent bio-signals simultaneously in many locations.

To address this need, optical or physical approaches have previously been employed. However, optical resolution suffers at depth, and physical probes, such as electrodes, can disturb the environment. Furthermore, parallel deployment of multiple probes to simultaneously record data from many cells remains uniquely challenging.

In particular, recording dynamical neural electrical activity in neurons has, over the past decade, been dominated by two kinds of technology. The first technology, electrodes, offers very temporally precise recordings of a sparse subset of the neurons within a region or regions, although some neurons are not recorded because the electrodes sample discrete points in space and because small or symmetrically shaped neurons may have small signals difficult to pick up by electrodes. Much ongoing effort aims to increase the number of electrodes deployable into a brain, increasing the number of neurons recorded, but not necessarily increasing the density of neurons recorded. The second, imaging of calcium dynamics, enables recordings of modest temporal resolution to be performed densely throughout small regions of the brain, but is limited by the need for the neurons to be near the surface of the brain to allow for microscopy accessibility, or by the need for an implanted optical device to monitor neural activity at depth.

Accordingly, there is a need in the art for further and improved systems and methods for recording dynamic neural activity, and the present disclosure fulfills that need.

### SUMMARY

Recording complex biological signals is a crucial application of synthetic biology, essential for a deeper understanding of biological processes. An ideal “biorecorder” would have the ability to record biological signals over a wide spatial distribution of cells with high temporal resolution. However, the biorecording tools available currently rely on transcription and translation of the biorecorder upon induction of the biological signal making their fastest possible temporal resolution ˜20 minutes.

The present disclosure provides a DNA polymerase-based biorecorder that can directly record environmental cationic concentration changes on to DNA in the form of nucleotide incorporation changes in the manner of a molecular ticker tape. Template-independent DNA polymerase, terminal deoxynucleotidyl transferase (TdT) can be used to add dNTPs somewhat randomly onto a single stranded DNA substrate, but that changes dNTP incorporation preferences in response to cations present in the extension reaction. The information stored in the DNA is readable, e.g., by sequencing the synthesized stand. The disclosure thus provides methods, systems, kits, and devices for recording condition changes or a sequence of condition changes, e.g., changes in an ionic environment over time, into a sequence of synthesized DNA.

For instance, in one aspect, the present disclosure provides methods of identifying or recording a biological signal comprising exposing a template-independent DNA polymerase to an organic environment comprising deoxyribonucleotide triphosphates (dNTPs) and a variable, allowing the DNA polymerase to transcribe a DNA substrate (i.e., add the dNTPs to the DNA substrate), and isolating the DNA substrate; wherein the dNTP content of the DNA substrate corresponds to the concentration of the variable in the organic environment.

In some embodiments, the template-independent DNA polymerase is a terminal deoxynucleotidyl transferase (TdT).

In some embodiments, the organic environment is the inside of a cell, such as a neuron. While in some embodiments, the organic environment is extracellular space between cells in a tissue or organ.

In some embodiments, the variable is a cation. In some embodiments, the cation may be selected from the group consisting of Co²⁺, Ca²⁺, and Zn²⁺.

In some embodiments, the DNA substrate is a single stranded DNA.

In some embodiments, the methods may further comprise sequencing the DNA substrate to determine the dNTP content of the DNA substrate. In some embodiments, sequencing the DNA substrate comprises next-generation sequencing (NGS), true single molecule sequencing (tSMS), 454 sequencing, SOLiD sequencing, ion torrent sequencing, single molecule real time (SMRT) sequencing, Illumina sequencing, nanopore sequencing, or chemical-sensitive field effect transistor (chemFET) sequencing.

In some embodiments, the method may further comprise determining the concentration of the variable based on the sequence of the DNA substrate. In some embodiments, the concentration is a relative concentration over time, while in some embodiments, the concentration is an absolute concentration over time. In some embodiments, determining the concentration comprises (a) reading the dNTPs on one strand and using a hidden Markov model to assign the most likely cation state at each base; or (b) reading the DNTPs of many strands in parallel, where at each time point, one base from each strand is used to estimate the incorporation frequency for that time point.

In another aspect, the present disclosure provides methods of detecting a change in a variable within a cell, comprising exposing a template-independent DNA polymerase within a cell to a variable, allowing the DNA polymerase to transcribe a DNA substrate, isolating the DNA substrate, and determining whether the concentration of the variable changed over time based on the sequence of the DNA substrate; wherein the dNTP content of the DNA substrate corresponds to the amount of the variable in the cell during transcription of the DNA substrate.

In some embodiments, the template-independent DNA polymerase is a terminal deoxynucleotidyl transferase (TdT). In some embodiments, the cell is a neuron.

In some embodiments, the variable is a cation. In some embodiments, the cation may be selected from the group consisting of Co²⁺, Ca²⁺, and Zn²⁺.

In some embodiments, the DNA substrate is a single stranded DNA.

In some embodiments, the methods may further comprise sequencing the DNA substrate to determine the dNTP content of the DNA substrate. In some embodiments, sequencing the DNA substrate comprises next-generation sequencing (NGS), true single molecule sequencing (tSMS), 454 sequencing, SOLiD sequencing, ion torrent sequencing, single molecule real time (SMRT) sequencing, Illumina sequencing, nanopore sequencing, or chemical-sensitive field effect transistor (chemFET) sequencing.

In some embodiments, determining whether the concentration of the variable changed over time comprises (a) reading the dNTPs on one strand and using a hidden Markov model to assign the most likely cation state at each base; or (b) reading the DNTPs of many strands in parallel, where at each time point, one base from each strand is used to estimate the incorporation frequency for that time point. In some embodiments, determining whether the concentration of the variable changed over time comprises determining the relative concentration of the variable over time. In some embodiments, determining whether the concentration of the variable changed over time comprises determining the relative concentration of the absolute over time.

The foregoing general description and following detailed description are exemplary and explanatory and are intended to provide further explanation of the invention.

### BRIEF DESCRIPTION OF THE DRAWINGS

FIG. 1 provides a device architecture of the disclosed TdT-based recording system (TURTLES) and its response to various environmental signals. (A) General device architecture and recording characteristic of DNA-editing based signal recorders. (B) General device architecture and recording characteristic of DNA synthesis based recorder. (C) General description of TdT-based untemplated recording of temporal local environmental signal (TURTLES). A time-varying input signal results in synthesis of ssDNA by TdT with varying dNTP compositions (shown as diagonal stripes for signal 0 and crisscross for signal 1). The various signals tested are shown as signal 1 and the background condition shown as signal 0.

FIG. 2 provides a depiction of one embodiments and how a DNA polymerase-based recorder can be different from currently available transcription/translation based DNA recorders.

FIG. 3 provides testing of the change in individual dNTP preference upon Co²⁺ addition. ssDNA substrate extensions carried out by TdT using just dATP, dTTP, dGTP, or dCTP in presence of Mg²⁺+Co²⁺ (first 4 lanes) or in presence of just Mg²⁺ (next 4 lanes) were run on a gel. “L” is ssDNA size marker. Reactions were carried out as mentioned in supplementary text.

FIG. 4 provides change in frequency of dATP, dCTP, dGTP and dTTP incorporation by TdT in the presence or absence of various signals. Signal 0 is always 10 mM Mg²⁺ at 37° C. for 1 hour. Signal 1 was, going from left to right: (1) 10 mM Mg²⁺+0.25 mM Co²⁺ at 37° C. for 1 hour; (2) 10 mM Mg²⁺+1 mM Ca²⁺ at 37° C. for 1 hour; (3) 10 mM Mg+20 μM Zn²⁺ at 37° C. for 1 hour; and (4) 10 mM Mg²⁺ at 20° C. for 1 hour. Error bars show two standard deviations of the mean. Statistical significance was assessed after first transforming the data into Aitchison space which makes each dNTP frequency change statistically independent of the others (see also FIG. 5).

FIG. 5 provides the length distribution of extensions upon addition of Co²⁺ based on NGS data. The mean frequency distribution of extension lengths was calculated for each condition (three biological replicates for each condition). Addition of Co²⁺ did not change the length distribution significantly.

FIG. 6 provides the length distribution of extensions upon addition of Zn²⁺ as seen on ssDNA gel. Extension reactions were run as mentioned in Materials and Methods section of Example 2. Two biological replicates per test condition were then loaded onto a ssDNA gel (Mg²⁺ on left and Mg²⁺+Zn²⁺ on right). Addition of Zn²⁺ increases the overall lengths of the extensions.

FIG. 7 provides the length distribution of extensions upon addition of Zn²⁺ based on NGS data. The mean frequency distribution of extension lengths was calculated for each condition (three biological replicates for each condition). Addition of Zn²⁺ caused a shift in probability distribution toward longer lengths.

FIG. 8 provides the length distribution of extensions upon addition of Ca²⁺ as seen on ssDNA gel. Extension reactions were run as mentioned in Materials and Methods section. Three biological replicates per test condition were then loaded on a ssDNA gel (Mg²⁺ on left and Mg²⁺+Ca²⁺ on right). Addition of Ca²⁺ decreases the overall lengths of the extensions.

FIG. 9 provides the length distribution of extensions upon addition of Ca²⁺ based on NGS data. The mean frequency distribution of extension lengths was calculated for each condition (three biological replicates for each condition). Addition of Ca²⁺ caused a shift toward shorter lengths.

FIG. 10 provides the length distribution of extensions upon using temperature as a signal based on NGS data. The mean frequency distribution of extension lengths was calculated for each condition (three biological replicates for each condition). Reducing the temperature of the extension reaction to 20° C. caused a shift toward shorter lengths.

FIG. 11 provides the mean % error in time prediction for 0→1 (Mg²⁺ to Mg²⁺+Co²⁺) data when different proportions of experimental data are used for time prediction (data is randomly sampled). To get an estimate about how the accuracy of time prediction will vary with the number of DNA sequences analyzed different proportions of experimental data obtained from the 0→1 setup were randomly sampled. Roughly 600,000 sequences were sequenced for each reaction. Good prediction is obtained when at least 6,000 (1% of the original data) sequences are used for each reaction. The mean extension length was roughly 15 bases (in 60 minutes) for all conditions.

FIG. 12 provides a recording of a single step change in Co²⁺ concentration onto ssDNA with minutes resolution in vitro. (A) Representative input unit step function used in our experiments by changing concentration of Co²⁺ from 0 mM to 0.25 mM during a TdT-based DNA synthesis reaction while keeping Mg²⁺ concentration and reaction temperature constant. (B) Expected step response of the TdT-based DNA recording system for the 0→1 input unit step function. (C) Experimental data for various input unit step functions each with 0.25 mM Co²⁺. Signal is calculated based on differences in dNTP preference. This plot shows there is a difference in the preference of dNTP incorporated by TdT in the Mg²⁺ (purple) and Mg²⁺+Co²⁺ (red) control conditions (where the signal (Co²⁺) is not added or removed throughout the extension reaction). The plot further shows the changes from 0→1 for Co²⁺ added at 10 minutes (blue), Co²⁺ added at 20 minutes (orange), and Co²⁺ added at 45 minutes (green). Total extension time for each of these experiments was 60 minutes. (D) Table showing the actual switch time as well as the mean inferred switch time along with each mean's standard deviation (mean calculated across 3 biological replicates).

FIG. 13 provides plots showing 0→1 data when different percentages of experimental data were randomly sampled. (A) 10% of sequences (roughly 60,000 reads) obtained from the NGS data for the 0→1 set-up were plotted for calculating switch times. (B) 1% of sequences (roughly 6,000 reads) obtained from the NGS data set for the 0→1 set-up were plotted for calculating switch times. (C) 0.1% of sequences (roughly 600 reads) obtained from the NGS data set for the 0→1 set-up were plotted for calculating switch times. (D) 0.01% of sequences (roughly 60 reads) obtained from the NGS data set for the 0→1 set-up were plotted for calculating switch times. It is important to note that sequences were chosen randomly. For reference see FIG. 12C, where 100% of the NGS data was plotted. Exact time predictions along with standard deviations can be found in Table 1.

FIG. 14 shows error in time predictions for each panel. The mean extension length was roughly 15 bases.

FIG. 14 provides dNTP bias & variability introduced by ssDNA wash columns. This figure provides a comparison of the composition of sequences retained when the extension reactions were directly used for ligation (“No Wash”) vs. when the same extensions were put through a ssDNA wash kit (“Wash”). (A), (B), (C) and (D) show individual plots of each nucleotide frequency seen in extension reactions between No Wash vs Wash conditions for just Mg²⁺ extensions. (E), (F), (G) and (H) show individual plots of each nucleotide frequency seen in extension reactions between No Wash vs Wash conditions for Mg²⁺+Co²⁺ extensions. A bias in overall dNTP content introduced by the columns used for ssDNA clean-up was observed when the reactions were washed after the recording experiment was stopped. ssDNA sequences with certain dNTP compositions were preferentially retained on the columns. (I) and (J) are plots for time prediction for No Wash and Wash condition respectively. An input signal of Co²⁺0→1 at 10 minutes for a 1 hour extension was used to obtained a time prediction of 12.8 minutes with 1.8 min std. dev. for No Wash condition. A time prediction of 12.4 min with a std. dev. of 1.2 min for the Wash condition was also obtained. While the time predictions were very similar, there is a clear increase in variability (std. dev.) for the later part of the signal recorded in (J) as compared to (I) (shown with a red arrow). Taken together, such biases and variability when introduced during the wash step for 0→1→0 experiment at 40 minutes for replacing +Co²⁺ buffers with −Co²⁺ buffers (see Materials and Methods: Extension reactions for 0→1→0 set-up of Example 2) would cause more noise for the final 20 minutes of the recording.

FIG. 15 provides data on the anomalous dNTP composition initially found at the end of reads and rate of reaction measured for extensions with only Mg²⁺ present. A significant change in the individual dNTP frequency towards the ends of the ssDNA sequences synthesized was observed. (A) Presents the significant change observed near the end of all reads with all the signals tested. Since we directly use 2 μL of extension reaction for ligation, the diluted TdT seems to be adding dNTPs to the ssDNA after the recording experiment, during the 16-hour ligation step. (B) To prove that these dNTPs were not added during the extension reaction (i.e. after the reaction), we sampled extension reactions (with Mg²⁺ only) at several time points (see Example 2). The mean extension length was calculated at each timepoint and applied a linear regression. The R² value of 0.96 for a straight line indicates that the assumption of constant rate (assuming input signal does not change) is valid. The slope of 0.17 reveals an average incorporation rate of 0.17 dNTPs/minute for this condition. Most importantly, the intercept of 5.82 indicates addition of 5.82 dNTPs (on average) either before or after the extension reaction. These are almost certainly being added after the extension reaction during the ligation step, which we conclude based on the anomalous behavior we see at the end of sequences in Panel A. (C) Plots of the data from Panel A were created after trimming off last few dNTPs. See Materials and Methods of Example 2 for details on how these 5.8 bases were trimmed from the end of all sequences before further analysis.

FIG. 16 provides recording multiple fluctuations of signal onto DNA. (A) Representative fluctuating input signal used in our experiments by changing concentration of Co²⁺ from 0 mM to 0.25 mM and back to 0 mM during a TdT-based ssDNA synthesis reaction while keeping Mg²⁺ concentration and reaction temperature constant. (B) Experimental data for fluctuating input signal of 0 mM Co²⁺→0.25 mM Co²⁺→0 mM Co²⁺ (010). Signal is calculated based on differences in dNTP preference. This plot shows there is a difference in the preference of dNTP incorporated by TdT in the Mg²⁺ (purple) and Mg²⁺+Co²⁺ (red) control conditions (where the signal (Co²⁺) is not added or removed throughout the extension reaction). The plot further shows the changes from 0→1→0 for Co²⁺ added at 20 minutes and removed at 40 minutes (blue). Total extension time for these experiments was 60 minutes. (C) Output fluctuating signal. Using the algorithm detailed in Glaser et al., the signal was deconvoluted into a binary response, with predicted switch times of 23.2 minutes and 40.7 minutes (actual: 20 minutes and 40 minutes). Signal predictions were made every 0.1 minutes and lines were added at the times of rise and fall of pulse for visualization.

FIG. 17 provides the total percent difference between dNTP preference changes at each position of synthesized strand under just 10 mM Mg²⁺ based TdT extensions in comparison to 10 mM Mg²⁺ plus 2 mM Ca²⁺, 10 mM Mg²⁺ plus 0.25 mM Co²⁺, 10 mM Mg²⁺ plus 20 μM Zn²⁺ based TdT extensions done in triplicates for a total extension time of 60 minutes. Individual percentage difference for each dNTP for each condition, A (blue), T (purple), C (green), G (red).

FIG. 18 provides a plot showing the difference in the preference of dNTP added at each length when TdT extensions take place without any Co²⁺, just Mg²⁺ (black); when Co²⁺ is added at time 0 (blue); when Co²⁺ is added at 10 min (orange); Co²⁺ is added at 20 min (green); or when Co²⁺ is added at 45 min (purple). Total extension time for these experiments was 60 minutes.

FIG. 19 provides a volcano plot depicting the various patterns of dNTPs for up to a length of 5 bases, indicating that identity of the last few bases affect the identity of the dNTP added and this preference changes in presence of Co².

### DEFINITIONS

The terminology used herein is for the purpose of describing the particular embodiments only, and is not intended to limit the scope of the embodiments described herein. Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a polymerase” is a reference to one or more polymerases and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “polymerase” refers to any enzyme which catalyzes the polymerization of ribonucleoside triphosphates (including deoxyribonucleoside triphosphates) to make nucleic acid chains. It is intended that the term encompass prokaryotic and eukaryotic polymerases, RNA and DNA polymerases, reverse transcriptases, high-fidelity and error-prone polymerases, thermostable and thermolabile polymerases, template-dependent and template independent polymerases, etc.

As used herein, the term “DNA polymerase” refers to an enzyme which catalyzes the polymerization of deoxyribonucleoside triphosphates to make DNA chains. In some embodiments, DNA polymerases use a nucleic acid template. Exemplary DNA polymerases that utilize a DNA template include prokaryotic family A polymerases (e.g., Pol I), prokaryotic family B polymerases (e.g., Pol II), prokaryotic family C polymerases (e.g., Pol III), prokaryotic family Y polymerases (e.g., Pol IV, Pol V), eukaryotic family X polymerases (e.g., Pol β, Pol λ, Pol σ and Pol μ), eukaryotic family B polymerases (e.g., Pol α, Pol δ, Pol ε, Pol ζ/Rev1), eukaryotic family Y polymerases (e.g., Pol η, Pol ι, and Pol κ), telomerase, eukaryotic family A polymerases (e.g., Pol γ and Pol θ), etc. DNA polymerases that are capable of utilizing an RNA template are “reverse transcriptases” (“RT”). Some RTs are also capable of utilizing DNA templates. Some polymerases, such as terminal deoxynucleotidyl transferase (“TdT) are template-independent, and indiscriminately add deoxynucleotides to the 3′ end of a nucleic acid strand.

As used herein, the term “oligonucleotide” (alternatively “oligo” or “oligomer refers to a molecule formed by covalent linkage of two or more nucleotides. Oligonucleotides are typically linear and about 5-50 (e.g., 5, 10, 15, 20, 25, 30, 35, 40, 45, 50, or ranges therebetween) nucleotides in length (although longer and shorter oligonucleotides may be within the scope of particular embodiments herein.

As used herein, the term “modified nucleotide” refers to nucleotides with sugar, base, and/or backbone modifications. Examples of modified nucleotides include, but are not limited to, locked nucleotides (LNA), ethylene-bridged nucleotides (ENA), 2′-C-bridged bicyclic nucleotide (CBBN), 2′,4′-constrained ethyl nucleic acid called S-cEt or cEt, 2′-4′-carbocycic LNA, and 2′ substituted nucleotides. Examples of base modifications include deoxyuridine, diamino-2,6-purine, bromo-5-deoxyuridine, 5-methylcytosine, and the like. Nucleotide modifications can also be evident at the level of the internucleotide bond, for example phosphorothioates, H-phosphonates, alkyl phosphonates, etc.; and/or at the level of the backbone, for example, alpha-oligonucleotides, polyamide nucleic acids (PMA), 2′-O-alkyl-ribonucleotides, 2′-O-fluoronucleotides, 2′-amine nucleotides, arabinose nucleotides, etc.

As used herein, the term “sequence identity” refers to the degree two polymer sequences (e.g., peptide, polypeptide, nucleic acid, etc.) have the same sequential composition of monomer subunits. For example, if oligonucleotides A and B are both 20 nucleotides in length and have identical bases at all but 1 position, then peptide A and peptide B have 95% sequence identity. As another example, if oligonucleotide C is 20 nucleotides in length and oligonucleotide D is 15 nucleotides in length, and 14 out of 15 nucleotides in oligonucleotide D are identical to those of a portion of oligonucleotide C, then oligonucleotides C and D have 70% sequence identity, but oligonucleotide D has 93.3% sequence identity to an optimal comparison window of oligonucleotide C. For the purpose of calculating “percent sequence identity” (or “percent sequence similarity”) herein, any gaps in aligned sequences are treated as mismatches at that position.

Any oligonucleotides described herein as having a particular percent sequence identity or similarity (e.g., at least 70%) with a reference sequence, may also be expressed as having a maximum number of substitutions (or terminal deletions) with respect to that reference sequence. For example, a sequence having at least Y % sequence identity (e.g., 90%) with SEQ ID NO:Z (e.g., 25 nucleotides) may have up to X substitutions (e.g., 2) relative to SEQ ID NO:Z, and may therefore also be expressed as “having X (e.g., 2) or fewer substitutions relative to SEQ ID NO:Z.”

As used herein, the term “hybridization” and linguistic variations thereof (e.g., hybridize) refers to the binding or duplexing (e.g., via Watson-Crick, Hoogsteen, reversed Hoogsteen, or other base pair formation) of a nucleic acid molecule (e.g., oligonucleotide (e.g., primer)) to a sufficiently-complementary nucleotide sequence (e.g., template) under suitable conditions, e.g., under stringent conditions.

As used herein, the term “stringent conditions” (or “stringent hybridization conditions”) refers to conditions under which an oligonucleotide (e.g., primer) will hybridize well to a perfectly complementary target sequence, to a lesser extent to less, but still significantly complementary sequences (e.g., 75% or greater complementarity), and not at all to, other non-complementary sequences.

As used herein, the term “complementary” (or “complementarity”) refers to the capacity for pairing between two nucleotides or nucleotide sequences with each another. Nucleic acid strands (e.g., primer and template) are considered “sufficiently complementary” to each other when a sufficient number of bases in the nucleic acids are capable of forming hydrogen bonds (e.g., with complementary bases) to enable the formation of a stable complex between the strands. To be stable in vitro or in vivo the sequence of an oligonucleotide need not be 100% complementary to its target nucleic acid. The terms “complementary” and “specifically hybridizable” imply that the nucleic acids bind strongly and specifically to each other to achieve a desired effect (e.g., priming of a template). Nucleic acid strands (e.g., primer and template) are considered “perfectly complementary” to each other when all of the bases in one nucleic acid strand are capable of forming Watson-Crick base pairs with a contiguous segment of the other nucleic acid.

As used herein, the term “sequencing” refers to any method of determining an order of nucleotides in a strand, and encompasses methods for determining the identity or character of a single nucleotide or a small number of nucleotides within a nucleic acid strand, and methods of determining an order or identity of nucleotides added or removed from a strand. A number of DNA sequencing techniques are known in the art, including fluorescence-based sequencing methodologies (See. e.g., Birren et al., Genome Analysis: Analyzing DNA. 1. Cold Spring Harbor. N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See. e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003. Analytical Biochemistry 320, 55-65; Shendure et al., 2005 Science 309, 1728-1732; U.S. Pat. No. 6,432,360. U.S. Pat. Nos. 6,485,944, 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246: herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), the Adessi PCR colony technology (Adessi et al. (2000). Nucleic Acid Res. 28, E87; WO 00018957; herein incorporated by reference in its entirety). and suitable combinations or alternatives thereof.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs and higher speeds in comparison to older sequencing methods. NGS methods can be broadly divided into those that require template amplification and those that do not.

Sequencing techniques that find use in some embodiments herein include, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into a sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), each of which is incorporated by reference in their entireties.

Another example of a DNA sequencing technique that finds use in some embodiments herein is 454 sequencing (Roche) (Margulies, M et al. 2005, Nature, 437, 376-380; incorporated by reference in its entirety). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments are attached to DNA capture beads, e.g., streptavidin-coated beads using, e.g., Adaptor B, which contains a 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that finds use in some embodiments herein is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that finds use in some embodiments herein is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982; incorporated by reference in their entireties). In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H⁺), which is detected and recorded in a sequencing instrument. The signal strength is proportional to the number of nucleotides incorporated.

Another example of a DNA sequencing technique that finds use in some embodiments herein is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a DNA sequencing technique that finds use in some embodiments herein is the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a DNA sequencing technique that finds use in some embodiments herein involves nanopore sequencing (Soni G V and Meller A. (2007) Clin Chem 53: 1996-2001; incorporated by reference in its entirety). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a DNA sequencing technique that finds use in some embodiments herein involves using a chemical-sensitive field effect transistor (chemFET) array to sequence DNA (for example, as described in US Patent Application Publication No. 20090026082; incorporated by reference in its entirety). In one example of the technique, DNA molecules can be placed into reaction chambers, and the template molecules can be hybridized to a sequencing primer bound to a polymerase. Incorporation of one or more nucleoside triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, other sequencing techniques (e.g., NGS techniques) understood in the field, or alternatives or combinations of the above techniques find use in some embodiments herein.

In some embodiments, the assays herein utilize single-molecule, highly-multiplexed, and/or high-throughput samples and techniques. In some embodiments, DNA barcoding of nucleic acid templates facilitates analysis of the substantial data collected in the assays herein. In certain embodiments, sequencing components that employ barcoding for labelling individual nucleic acid molecules are employed. Examples of such barcoding methodologies and reagents are found in, for example, U.S. Pat. Pub. 2007/0020640, U.S. Pat. Pub. 2012/0010091, U.S. Pat. Nos. 8,835,358, 8,481,292, Qiu et al. (Plant. Physiol., 133, 475-481, 2003), Parameswaran et al. (Nucleic Acids Res. 2007 October; 35(19): e130), Craig et al. reference (Nat. Methods, 2008, Oct. 5 (10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO2014/020127; all of which are herein incorporated by reference in their entireties, including for reaction conditions and reagents related to barcoding and sequencing of nucleic acids.

### DETAILED DESCRIPTION

DNA is an outstanding medium for information storage. However, to date, the ability to record de novo information into it has been limited. The present inventors recognized that if facile methods for recording temporal information (i.e., the change in a signal over time) into DNA at the rate that DNA polymerases synthesize DNA could be developed, it would revolutionize the ability to investigate neural activity in the brain, developmental biology, and other microscopic biological phenomena where scale (simultaneously record millions of cells), spatial resolution (individual recordings at the single cell or subcellular level), and temporal resolution (subsecond sampling frequency) are limited by current technology. Outside of biology, DNA is a promising medium for certain data storage problems, surpassing magnetic, optical, and solid-state hard drives currently used for information density.

Additionally, recording biological signals (i.e., bio-signals) can be difficult in three-dimensional matrices, such as tissue. The present disclosure presents a DNA polymerase-based strategy that records temporal bio-signals locally onto DNA to be read out later, which can obviate the need to extract information from tissue on the fly or in real time. The disclosed processes utilize a template-independent DNA polymerase (e.g., terminal deoxynucleotidyl transferase (TdT)) that probabilistically adds dNTPs to single-stranded DNA (ssDNA) substrates without a template. In vitro, the dNTP-incorporation preference of TdT changes with the presence of Co²⁺, Ca²⁺, Zn²⁺ and temperature. Extracting the signal profile over time is possible by examining the dNTP incorporation preference along the length of synthesized ssDNA strands like a molecular ticker tape. In some embodiments, this TdT-based untemplated recording of temporal local environmental signals may be referred to as “TURTLES”. The present disclosure shows that the disclosed methods can determine the time of Co²⁺ addition (or other bio-signal) to within two minutes over a 60-minute period. Further, TURTLES has the capability to record multiple fluctuations. This allows for the estimation of the rise and fall of an input signal (such as a Co²⁺ pulse) to within three minutes. TURTLES has at least 200-fold better temporal resolution than all previous DNA-based recording techniques.

Thus, provided herein are systems and methods for using DNA polymerases to record information onto DNA for single cell high time-resolution recording and for high density data storage. The technology provides a DNA polymerase-based nanoscale device that can be genetically encoded to record temporal information about the polymerase's environment into an extending single strand of DNA. As a signal changes in time, the nucleotides incorporated also change, such that a strand of DNA encodes a “ticker-tape.” As the recorder is a DNA polymerase, it can be genetically encoded into any cell line, allowing for expression and recording across large tissues and organs. DNA sequencing can be done at low cost, allowing the retrieval of massive amounts of information. By way of example and not as a limitation, the technology finds use in single cell signal recording of cations for neuroscience and developmental biology, studying changes in concentration of other biologically important cations in neurons and other organs, and for information data storage in general. Compared to existing systems of recording biological information, the technology provided herein is much smaller in size (nanometers as opposed to millimeter size of most current neural imaging technology); it can store a signal locally at a single neuron level, which no technology can currently offer; it has demonstrated temporal resolution for signal recording of up to 1 minute (and theoretically could achieve sub-second resolution), it is highly adaptable, e.g., for recording several different cations; and it is extendable to other environmental signals.

Current technologies rely on phosphoramidite synthesis, which has 1 base resolution but is relatively slow. The technology of the invention can readily incorporate at least one base per second (compared to 1 base per 20 min in a phosphoramidite cycle), and may achieve 1 bit per 5-10 bases.

Biological signaling is of equal importance for the propagation of a single cellular organism as it is in the functioning of a complex multicellular organism. These signals can be in the form of ionic fluctuations, small molecule metabolite variations and DNA, RNA, peptide or protein expression/inhibition. Moreover, they can occur for different time scales, from milliseconds to hours with varied spatial distribution, from within a single cell to between several millions of cells at once. Being able to study such biological signaling events at high spatial and temporal resolution is thus a critical challenge.

With the steady decrease in DNA sequencing costs and several attempts to commercialize it as a data storage medium, being able to leverage DNA for biotic signal recording is an ideal solution to the problem. Information stored in DNA can be stably preserved for long periods of time. Moreover, advances in next-generation sequencing make it easy to precisely decode information stored in DNA in a cost-efficient and fast manner.

Several attempts have been made at recording biological signals onto DNA in living cells. Recombinase-based techniques RSM and BLADE utilize the interaction of a sensor with the biological signal resulting in expression of different recombinases, which then target specific addresses in the genome of the cell and record orthogonal signals over several cell generations.

SCRIBE and mSCRIBE involve the expression of ssDNA or RNA in response to the biosignal and this single stranded nucleotide then results in editing of either a targeted or untargeted DNA sequence. Single base editing can also be used for signal recording as described in CAMERA, which also involves the interaction of a biosensor with the signal, resulting in transcription and translation of the DNA recorder that, in a directed manner, is able to convert C•G to T•A.

GESTALT, MEMOIR, TRACE, and Shipman and Kalhor's techniques all creatively use bio-signal induced Cas9 expression for targeted in vivo DNA editing.

While all of these methods are excellent for several specific recording applications, they are limited in time-resolved signal recording over small time intervals. Most, if not all of these “biorecorders” involve the signal resulting in activation of the transcription and translation machinery, making the fastest possible recording timescales about 20 minutes. Moreover, due to the nature of their applications, they have been optimized for recording at a population level and, as such, lack high spatial resolution.

Some of the fastest signaling events happen during neural synapses. Thus, functional connectome analysis of the brain relies heavily on studying such signal generated by calcium concentration changes, or voltage changes happening at millisecond timescales in various neurons.

Imaging of calcium dynamics enables recordings of modest temporal resolution to be performed densely throughout small regions of the brain, but is limited by the need for the neurons to be near the surface of the brain for microscopy accessibility purposes, or by the need for an implanted optical device to monitor neural activity at depth. Genetically encoded biorecorders (nanoscale biological devices that record biosignals), specifically those that store information in DNA, represent an attractive alternative. These biorecorders could be delivered to all cells through transgenesis where they are synthesized locally and record in parallel, obviating the challenges of optical and physical methods that must recover the data on the fly across many cells and in deep tissues.

To overcome dependence on macroscopic devices, a number of new technologies propose to encode neural activity in a non-invasive chemical form. Every cell encodes its own neural activity in a lasting form that can be later read out via anatomical or chemical means post hoc. The genetically engineered tool CAMPARI, for example, is a fluorescent protein that undergoes a green-to-red transition when illuminated in presence of calcium. The genetically encoded tools FLARE and Cal-Light sense the coincidence of elevated calcium and illumination to trigger gene expression, similarly capturing temporally-strobed calcium level into an enduring transcriptional change. However, despite much ongoing excitement and utilization of these tools, each of these technologies can only capture neural activity at one time-point, raising the question of whether a time series of neural activity could be recorded into a molecular form, in the fashion of a ticker tape.

The feasibility of a DNA polymerase-based cation concentration recorder has previously been analyzed. Several reviews have highlighted the advantages of a molecular ticker-tape over other currently available techniques. Neural application of such a recorder would be the most advanced one, apart from that there are several other cations that play significant role as secondary messengers in neurons and other cells. The only limitation of this application is having a DNA polymerase with biochemical parameters that make it suitable for such recordings.

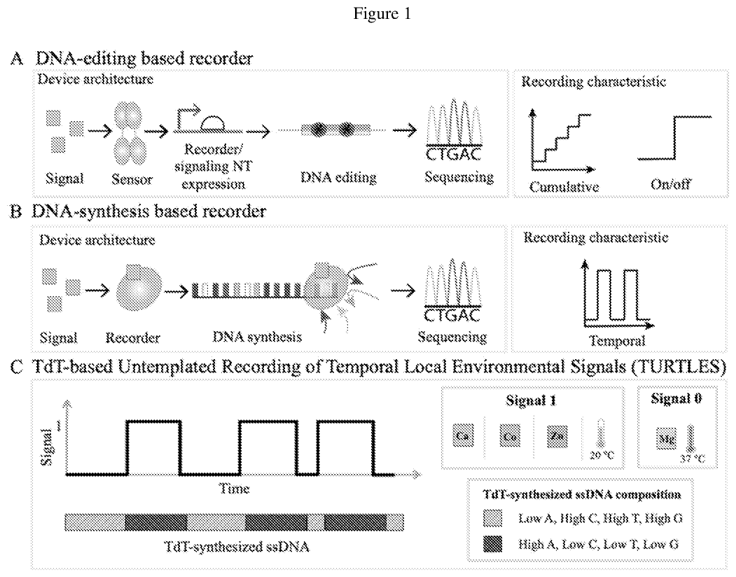

To date, biorecording strategies that record onto DNA locally and are genetically encodable have been demonstrated with temporal resolution of two hours or more. These DNA-editing based techniques primarily rely on nucleases or recombinases, both of which are limited to a temporal resolution on the scale of hours because of the time required for (a) expression of the DNA-modifying enzyme and (b) DNA cleavage and repair to store the data. Moreover, due to the architectures of these recording devices, signals are recorded in a cumulative (or on/off) fashion (FIG. 1). Cumulative signals can determine the amount of a signal a biorecorder was exposed to, but not the specific times of exposure. It is important to deliver bio-signal measurements with higher temporal resolution and higher information content.

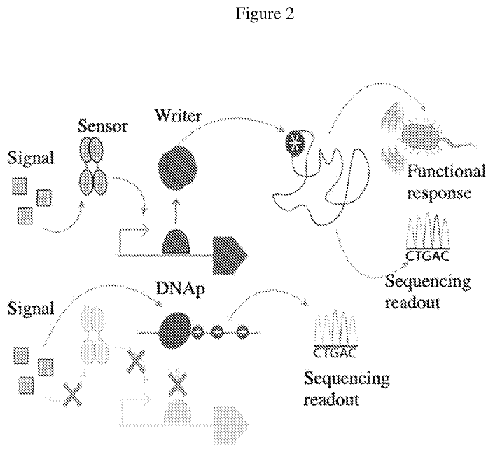

The technology provided herein converts a DNA polymerase into a biorecorder, such that there is no need for intermediated steps of signal-dependent induction and resulting transcription and/or translation (FIGS. 1 and 2). Essentially, the DNA polymerase-based recorder acts like a molecular ticker tape, where the identity of the nucleotide added to the DNA strand depends on the biological signals in the environment. Since DNA polymerases synthesize DNA at a fast rate, this makes possible recording several environmental fluctuations that occur on a minute's timescale on to DNA. Moreover, since the record of the bio-signal is a DNA molecule, it can be easily barcoded for single cell spatial resolution.

The present technology provides a template-independent polymerase, terminal deoxynucleotidyl transferase (TdT), so that the record produced is a de novo sequence, not governed by any template nucleic acid molecule. Terminal deoxynucleotidyl transferases (TdTs) belong to a unique class of DNA polymerases (DNAp) that synthesize single stranded DNA (ssDNA) in template-independent fashion. TdTs incorporate dNTPs probabilistically to the 3′ termini of ssDNA substrates according to an inherent dNTP incorporation preference. As shown herein, this dNTP preference is affected by changes in the TdT reaction environment. When the dNTP incorporation preference is altered, then information about the environment could be recorded in each incorporated dNTP. Thus, the disclosed systems and methods provide a DNA-synthesis based biorecorder for achieving the spatiotemporal resolution that eludes the current DNA-editing based biorecorders.

Thus, the disclosed processes and methods leverage TdT's natural tendency to alter preference for dNTPs based on the cations present in its environment. During development of the technology, the percentage change in preference of the TdT for incorporating the 4 different dNTPs upon change in its cationic environment was quantified. The size of a step change in a cation concentration that could be successfully recorded on to DNA was initially estimated, and based on the estimation, the technology was able to successfully record a step change of 10 minutes time-scale with a resolution of about 1 minute. The technology successfully recorded 15 signal fluctuations of 4 minutes each on the same DNA strand. While an embodiment of a recorder provided herein is well-suited for Co⁺ concentration recording, the technology is not limited to this cation, is adaptable for use with calcium and other cations.

Because TdT is a template-independent DNA polymerase and the identity of the incoming base is not determined by complementation to a template strand, the nucleotide that is incorporated is a random process. This random process is biased. For example, under standard in vitro conditions, TdT will incorporate 24.5% A, 15.0% C, 45.3% G, and 15.2% T. The present technology has recognized that fact that the frequency at which a base can be incorporated can be leveraged to produce a biorecorder. In embodiments disclosed herein, the technology uses the property of TdT that the divalent cation present in the reaction mixture shifts the frequency of bases incorporated. By reading the DNA sequence of the strand synthesized by TdT, the cation concentration present at the time those bases were incorporated can be estimated.

Because it is a probabilistic process, a plurality of observations of each nucleotide position are generally required to determine the incorporation frequency and to correctly assign the cation concentration that is consistent with those observations. This can be accomplished, for example, by (a) reading many nucleotides on one strand, in conjunction with the use of hidden Markov models to assign the most likely cation state at each base; or (b) reading the nucleotide of many strands in parallel, where at each time point, one base from each strand is used to estimate the incorporation frequency for that time point.

While the discussion herein has focused on the embodiment of measuring temporal cation concentrations (Co²⁺, Ca²⁺, Zn²⁺), it is contemplated that the frequency of base incorporation can be manipulated by many other environmental variables. As with cations, it is contemplated that a number of environmental variables can be recorded by wild-type TdT (e.g., temperature, pH, surfactant concentration). It is further contemplated that protein engineering may be used to create modified TdT molecules, e.g., chimeras or conjugates that incorporate protein domains that change conformation when bound by a specific ligand (e.g., maltose binding protein). Such modified TdT polymerases find application in the present technology, e.g., by altering the TdT structure in response to the conformational change, the base incorporation frequency and/or the incorporation rate may change, reflecting, for example, the time of the binding event. In some embodiments, a plurality of TdTs that have different base incorporation frequencies may be used in parallel. By modulating the activity of one (or more) of the plurality of TdTs, the relative incorporation frequencies may be used to determine the activity ratio(s) of the TdTs at different points in the extension.

The present disclosure provides, in some embodiments, methods of TdT-based Untemplated Recording of Temporal Local Environmental Signals (TURTLES). These methods can achieve minute-scale temporal resolution (a 200-fold improvement over existing DNA recorders) and outputs a truly temporal (rather than cumulative) signal. Changes in divalent cation concentrations (Ca²⁺, Co²⁺, and Zn²⁺) and temperature alter dNTP incorporation preferences of TdT and that concentrations and temperatures can be recovered by analyzing the ssDNA synthesized by TdT. Thus, temporal information can be obtained by using estimates of dNTP incorporation rates, allowing us to map specific parts of a DNA strand to moments in time of the recording experiment. Using this approach, temperature and divalent cation dynamics can be recorded with a few minutes frequency. The Examples below demonstrate the utility of TdTs as DNA-based biorecorders with high temporal resolution.

Indeed, the results shown herein indicate that TURTLES (and other template independent systems) can record temporal changes in divalent cationic concentrations and temperature onto DNA at minutes timescale resolution in vitro. The methodology presented here is two orders-of-magnitude faster than any of the currently utilized DNA-based environmental signal recording techniques. This enhancement in temporal resolution is because the disclosed biorecorder does not rely on temporal expression of DNA-modifying enzymes or DNA repair processes and is simply limited by (a) the incorporation rate of TdT, which is 1 dNTP per second under optimal conditions and (b) the magnitude of the dNTP incorporation preference change. Because this recording system can fully switch from one state and back to the original, the information recording is truly temporal instead of cumulative, unlike nuclease/recombinase based recording techniques.

As with all DNA-based recording schemes, TURTLES (and other template independent systems) can be encoded genetically, and be employed to record and store information locally in DNA with single cell resolution in tissues, where recovering information in real time is challenging via optical or electronic approaches. Adding a unique barcode to each cell being studied can simplify recovery of spatial resolution. Moreover, based on previous calculations of the metabolic burden on a cell expressing such a de novo DNA recording system; given its current signal recording capability and resolution would make recording 10s of temporal events in a single experiment metabolically feasible.

The disclosed methods and processes can also reduce the cost of DNA synthesis associated with phosphoramidite chemistry. In vitro TdT-based recorders could allow the storage of arbitrary digital information into DNA by controlling the environment to record ‘1s’ and ‘0s.’ For example, a low temperature could be ‘0’ and a high temperature could be ‘1’. Indeed, at least a 1 bit per 10 base resolution is possible based on the disclosed methods. As such, TUTRLES could provide a cheaper more environmentally friendly option for DNA data storage.

Template independent-based DNA recording is a promising technology for interrogating biological systems, such as the brain, where high temporal and spatial resolution is needed. In such systems measurement across many cells are required, and the depth of tissue prevents extracting measurements on the fly from using physical and optical methods. Thus TURTLES provides many exciting opportunities for recording complex biological processes that were previously infeasible.

TdT can Detect Environmental Signals In Vitro Via Changes in dNTP Incorporation Preference

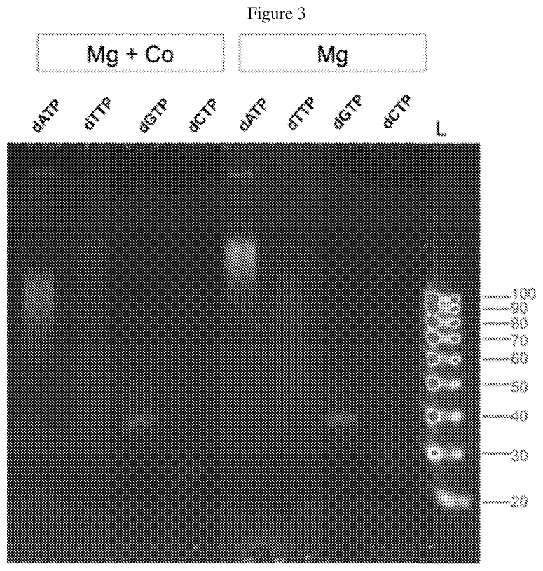

For TdT, the kinetics of incorporation for specific nucleotides is affected by the cations present in the reaction environment. For example, when only one nucleotide is present, TdT incorporation rates of pyrimidines, dCTP and dTTP, increase in the presence of Co²⁺ (FIG. 3).

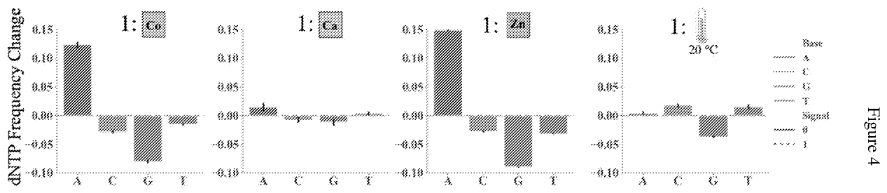

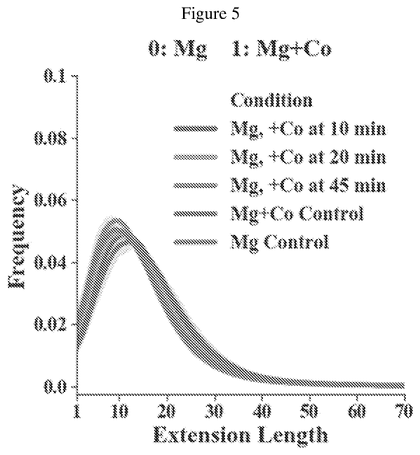

Co²⁺-dependent changes in kinetics occur in the presence of all four nucleotides, dATP, dCTP, dGTP, and dTTP (hereon referred to as A, C, G, and T). ssDNA substrate extended by TdT in the presence of Mg²⁺ only and with 0.25 mM CoCl₂ added were determined by single molecule sequencing. Upon Co²⁺ addition, A incorporation increased by 13%, while G decreased by 10% and T and C decreased by 3 and 2 percent respectively (these values do not sum to 0% due to rounding error) (FIGS. 4 and 5). This shift in dNTP incorporation preference could be used to determine if Co²⁺ was present or not during ssDNA synthesis.

Ca²⁺, Zn²⁺, and temperature fluctuations can also be recorded by the disclosed systems. Ca²⁺ is a proxy for neural firing, Zn²⁺ is an important signal in development and differentiation of cells, and temperature is relevant in many situations.

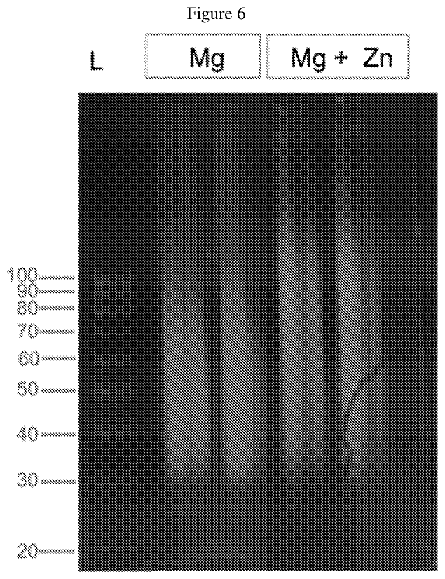

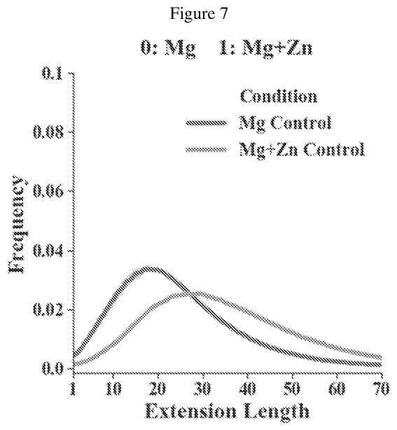

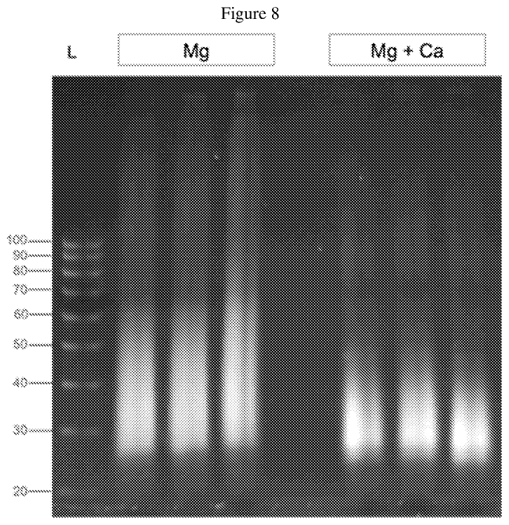

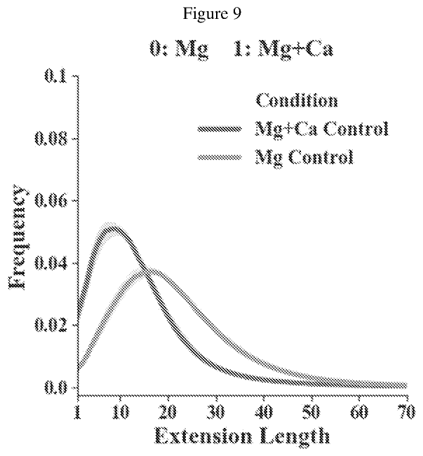

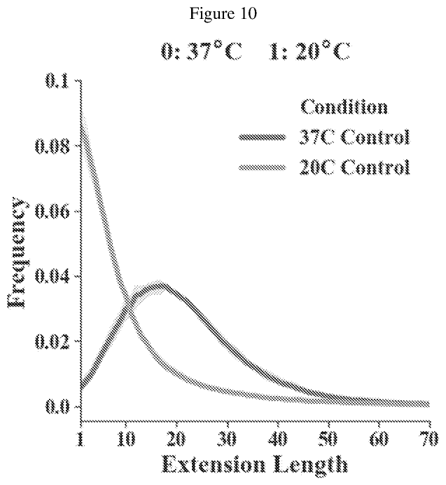

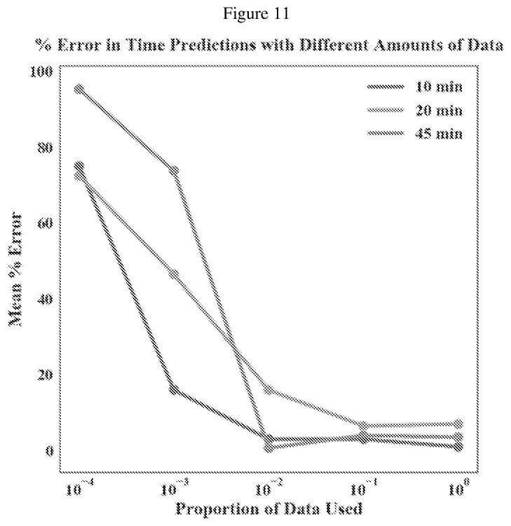

Different environmental signals had differences both in the particular dNTP affected and the magnitude of the dNTP incorporation preference change. For instance, 20 μM Zn²⁺ provided a 15% increase in a preference for A, 8% decrease in a preference for G, 4% decrease in a preference for T, and 3% decrease in a preference for C (FIGS. 4 and 5). dNTP incorporation preference upon 1 mM Ca²⁺ addition changed more modestly. The change was 1.4% increase in A, 1.7% decrease in G, 1.0% increase in T and 0.5% decrease in incorporation of C (FIGS. 4 and 5). Finally, reaction temperature was changed from the preferred 37° C. to 20° C. and this produced a 3% increase in A, 3.5% decrease in G, 1.0% increase in T and 0.5% decrease in incorporation of C (FIGS. 4 and 5). The addition of cations as well as temperature change altered the dNTP incorporation rates and lengths of ssDNA strands synthesized (FIGS. 6-11). Thus, the effect of multiple biologically relevant signals (i.e., bio-signals) were able to be characterized and recorded with TdT activity. Further analysis of TURTLES focuses on Co²⁺ as the candidate cationic signal for exemplary purposes only.

Recording a Single Step Change in Co²⁺ Concentration onto DNA with Minutes Resolution In Vitro

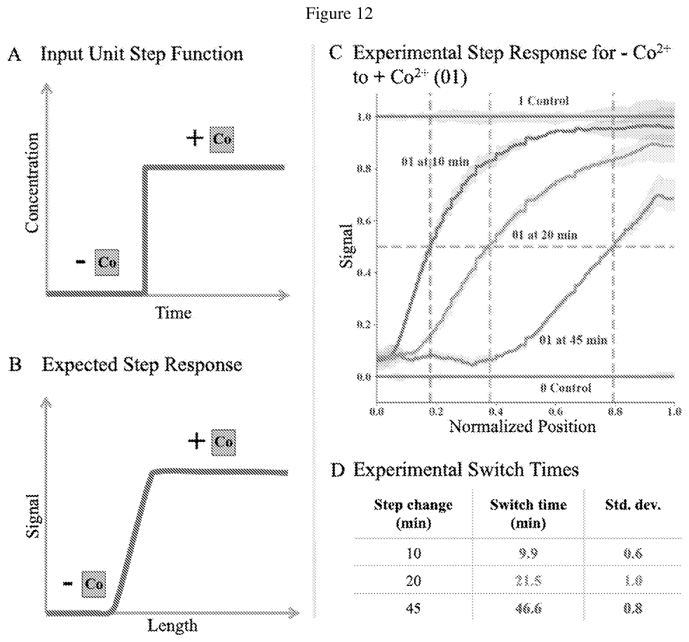

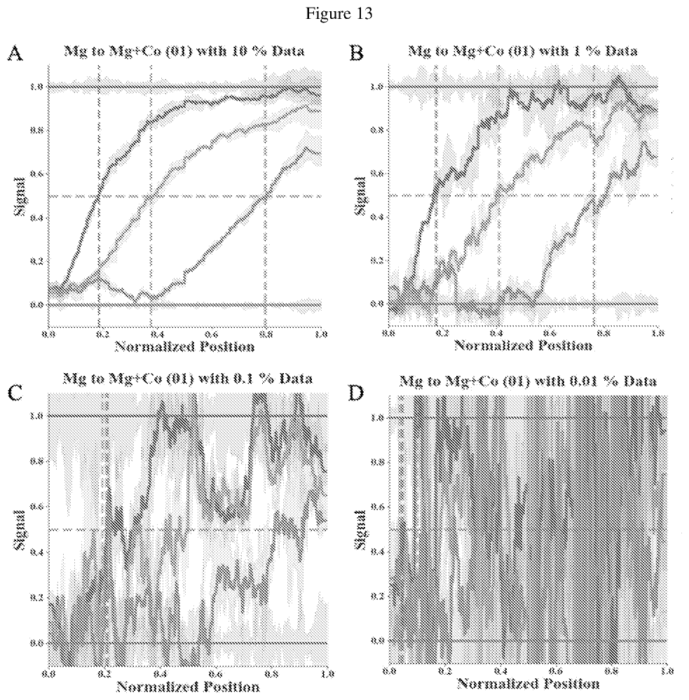

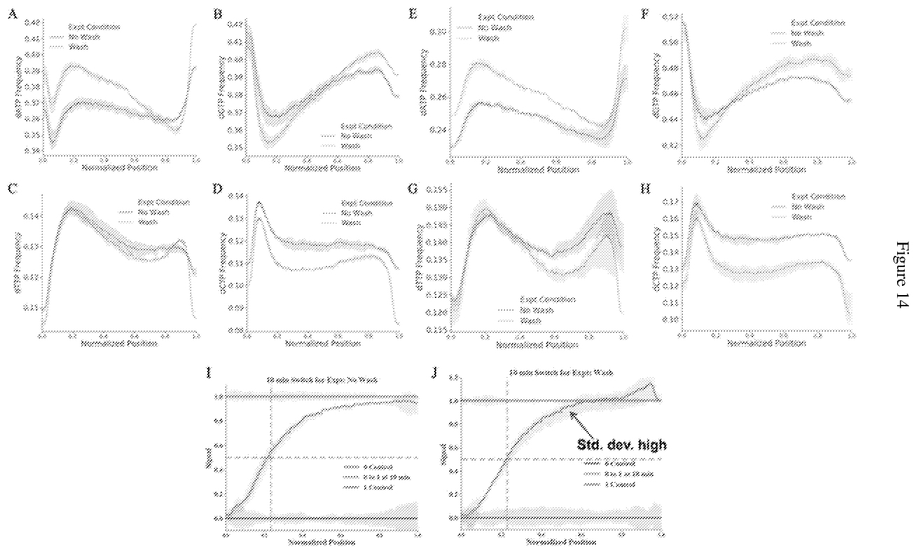

Having quantified the distinct change in dNTP incorporation preference upon Co²⁺ addition, the time at which Co²⁺ was added to a TdT-catalyzed ssDNA synthesis reaction was examined based on the change in sequence of the synthesized ssDNA strands (FIGS. 12 A and B). During a 60 min extension reaction, input unit step functions were created at 10, 20, and 45 minutes by adding 0.25 mM Co²⁺ at those times (this is referred to as a 0→1 signal where ‘0’ is without Co²⁺ and ‘1’ is with 0.25 mM Co²⁺). This was done to infer specific times from the DNA readout. For each reaction, approximately 500,000 DNA strands were analyzed by single molecule sequencing and calculated the dNTP incorporation frequencies over all reads. By plotting the change in dNTP incorporation frequency along the extended strands after normalizing each sequence by its own length, the results indicated that later addition of Co²⁺ resulted in changes farther down the extended strand (FIG. 12C). The average location across all the sequences was then calculated for a given condition at which half the 1 control (Mg²⁺+Co²⁺) signal was reached. To translate this location into a particular time in the experiment, a constant rate of dNTP addition was assumed (FIG. 13) and an equation was derived that adjusted for the change in rate of DNA synthesis between the 0 and 1 controls (Equation 5, Materials and Methods of Example 2). Using this information, the Co²⁺ additions could be estimated to be at 9.9, 21.5 and 46.6 minutes (FIG. 12D). This data also enabled the estimation of the time within 7 minutes of the unit input step function for the reverse; a change in signal (Co²⁺ concentration) from 1 to 0 (FIG. 14). Thus, TURTLES has excellent temporal precision, approximately 200-fold higher than any other currently utilized biorecorders.

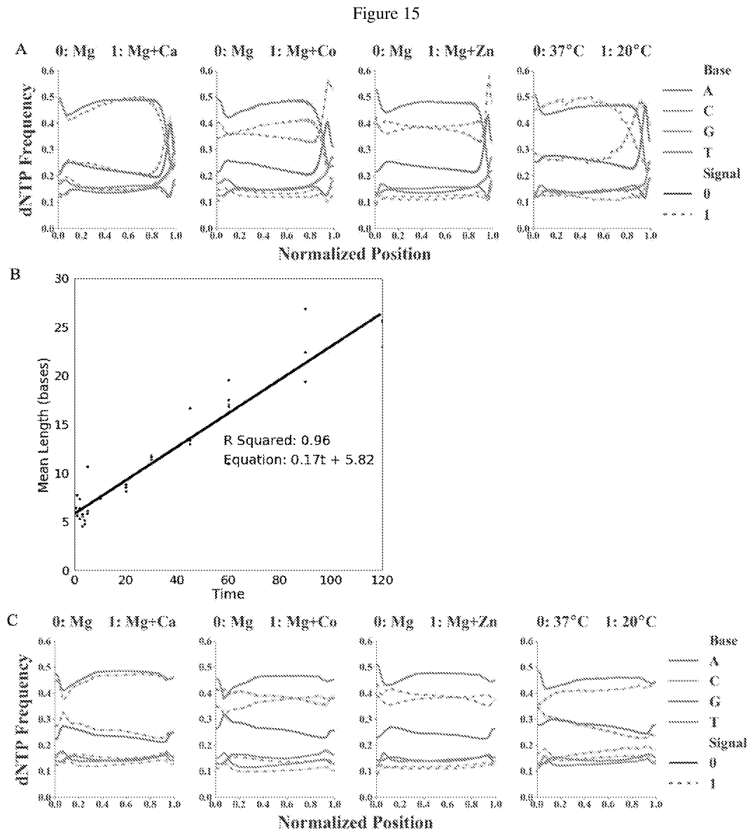

While this method allowed for the accurate estimation of the times of Co²⁺ addition (0→1) and removal (1→0), in many applications, simultaneously synthesizing ˜500,000 strands of DNA would be infeasible. To determine the number of strands needed for reasonable statistical certainty, smaller groups of strands from the experiment were randomly sampled and evaluated for the ability to predict when Co²⁺ was added (FIG. 15). With only ˜6,000 strands, the time of Co²⁺ additions was still estimated to be at 9.7, 23.2 and 44.7 minutes, as shown in Table 1 below. Thus, even with a limited number of strands, high temporal precision recording is feasible.

TABLE 1

| | | | | Expt | | | | Pro- |
|---|---|---|---|---|---|---|---|---|
| | Percent | Average | Actual | Mean | | | | portion |
| | of | # reads | Switch | Switch | | | | of |
| | Data | per | Time | Time | Std | % | Average | Data |
| | Used | replicate | (min) | (min) | Dev | Error | % | Used |
| 0 | 100 | 588,000 | 10 | 9.9 | 0.6 | 1 | 3.85 | 1 |
| 1 | 100 | 588,000 | 20 | 21.4 | 1.1 | 7 | 3.85 | 1 |
| 2 | 100 | 588,000 | 45 | 46.6 | 0.8 | 3.6 | 3.85 | 1 |
| 3 | 10 | 58,800 | 10 | 10.3 | 0.2 | 3 | 4.5 | 0.1 |
| 4 | 10 | 58,800 | 20 | 21.3 | 1.7 | 6.5 | 4.5 | 0.1 |
| 5 | 10 | 58,800 | 45 | 46.8 | 1.6 | 4 | 4.5 | 0.1 |
| 6 | 1 | 5,880 | 10 | 9.7 | 0.6 | 3 | 6.56 | 0.01 |
| 7 | 1 | 5,880 | 20 | 23.2 | 1.6 | 16 | 6.56 | 0.01 |
| 8 | 1 | 5,880 | 45 | 44.7 | 2.2 | 0.7 | 6.56 | 0.01 |
| 9 | 0.1 | 588 | 10 | 11.6 | 0.4 | 16 | 45.43 | 0.001 |
| 10 | 0.1 | 588 | 20 | 10.7 | 5.2 | 46.5 | 45.43 | 0.001 |
| 11 | 0.1 | 588 | 45 | 11.8 | 0.3 | 73.8 | 45.43 | 0.001 |
| 12 | 0.01 | 59 | 10 | 2.5 | 1 | 75 | 80.94 | 0.0001 |
| 13 | 0.01 | 59 | 20 | 5.5 | 2.3 | 72.5 | 80.94 | 0.0001 |
| 14 | 0.01 | 59 | 45 | 2.1 | 2.1 | 95.3 | 80.94 | 0.0001 |

To get an estimate about how the accuracy of time prediction will vary with the number of DNA sequences analyzed different proportions of experimental data obtained from the 0→1 setup were randomly analyzed. Roughly 600,000 sequences were sequenced for each reaction. Good prediction is obtained when at least 6,000 (1% of the original data) sequences are used for each reaction with a standard deviation of about 1.4 minutes.

Recording Multiple Fluctuations in Co²⁺ Concentration onto DNA with Minutes Resolution In Vitro

An advantage of this approach is that it can record the time of multiple fluctuations. This is in contrast to any of the other DNA-based recorders, which rely on an accumulation of signal (i.e., accumulation of mutations). Accumulation can tell what fraction of the time a signal was present over a period of time, but not how the signal was distributed throughout the time period of recording. The ability to know when fluctuations occur adds new levels of insight into different biological systems.

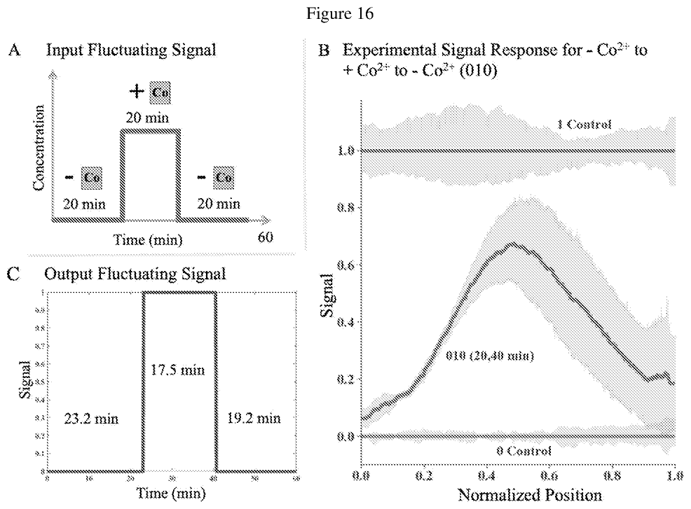

The disclosed TURTLES system was used to record a 0→1→0 signal, where ‘0’ is without Co²⁺ and ‘1’ is with 0.25 mM Co²⁺ (FIG. 16A). The signal was 0 for the first 20 minutes, 1 for the next 20 minutes, and 0 for the last 20 minutes of the extension reaction (FIG. 16A). The sequencing data obtained from the experiment was used to calculate the signal (FIG. 16B). Because multiple step changes were present, an algorithm discuss in the Materials and Methods for Example 2 (see “Timepoint prediction for 0→1→0 multiple fluctuations experiment”) was used to estimate the true value of the signal at all times (every 0.1 min). The signal reconstruction clearly resembles the true 0→1→0 signal, with transitions between the 0 and 1 signals occurring at 23.2 and 40.7 minutes (FIG. 16C). Finally, using in silico simulations based on the experimental parameters of TdT, it is clear that a TdT-based recording system can accurately record more than 3 pulses and pulses of much shorter duration than 20 minutes. Overall, this demonstrates the capability of TURTLES to record multiple temporal fluctuations.

Co²⁺ Affects TdT's Preference of dNTP Incorporation in Mg Background

Unlike canonical DNA polymerases, TdTs can utilize at least four different cations for DNA synthesis. Also, TdT activity is notably more sensitive to the local environment, including the specific cations present in the reaction mixture. With Mg²⁺ these enzymes have been shown to have biases for which dNTP is incorporated as follows: dGTP>dCTP>dTTP>dATP. Previous studies have shown that Co²⁺ addition increases the catalytic polymerization efficiency of pyrimidines, dCTP and dTTP, which was confirmed in the development of the present technology. However, none of those studies tested the change in the catalytic activity of TdTs in presence of all the dNTPs. Since the ideal application of this biorecorder would be inside a living cell where all dNTPs will be present. Thus, during the development of the instant technology, quantifying the change in nucleotide preference in conditions where all dNTPs were present was examined.

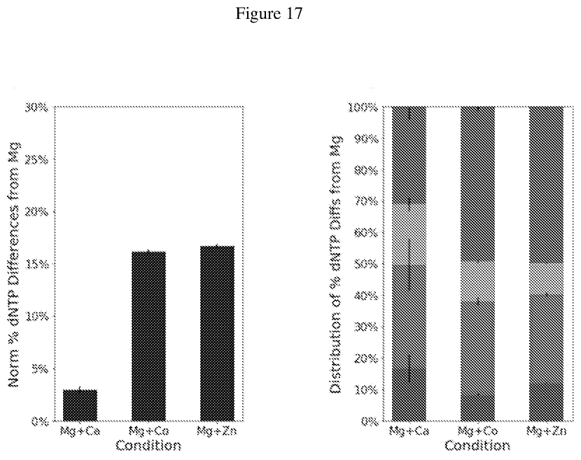

Previously developed next generation sequencing (NGS) methods were adapted for template-independent DNA polymerases to compare effects of different cations on dNTP preference. Measures were taken to ensure that the data analyzed was not biased by PCR amplification. The bio-signal of Co going from zero to 0.25 mM in a 10 mM Mg background was examined. Upon Co addition, a 13% increase in A was observed, while 10% decrease in G and 3 and 2 percent decrease in T and C respectively was observed (FIG. 17). Overall an approximately 15% change was measured between the two conditions (FIG. 17).

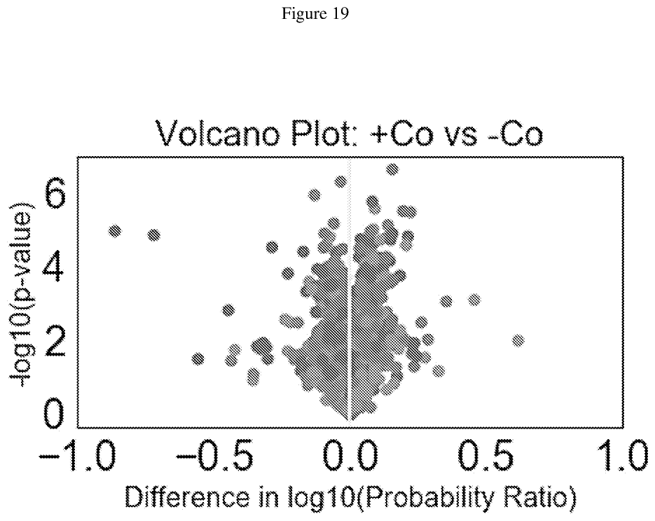

How the composition of the primer affects the identity of the nucleotide added was examined. For doing this analysis, the effect of up to the last five bases in the primer was examined. It was determined that only the identities of the last four bases were catalytically relevant (FIG. 19).

Recording Single Step Change in Co²⁺ Concentration with a Minute Resolution on to DNA:

Reactions were next examined to determine whether the time at which Co was added to an extension reaction could be identified based on the change in dNTP distribution of the synthesized DNA strands. This Co addition was defined as a single step change. The standard deviation in the predicted time as compared to the known time of Co addition was defined as step-response time of the recording system.

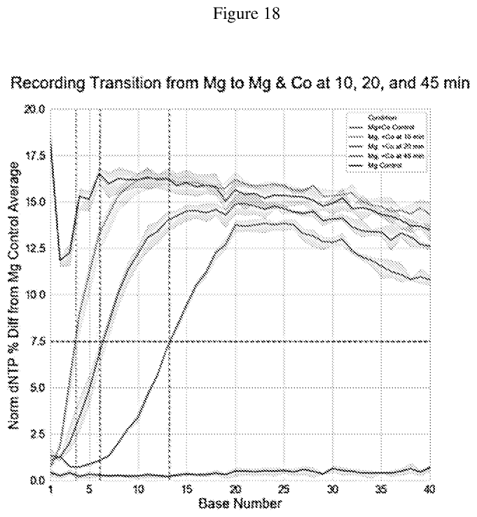

Measurements were taken to determine how small of a step-change in signal could be recorded on to DNA by changing the cation concentrations. Three different times of Co addition were tested: 10 minutes, 20 minutes and 45 minutes. The rate of dNTP addition was then estimated based on the total length of the experiment and the total number of nucleotides added in each reaction. The length of the synthesized strands was plotted against the percentage of each dNTP at each position (FIG. 18). For these curves, it was estimated that the length at which the % of dNTPs changed half of the total difference between the Mg only or the Mg+Co condition (12.5%). These lengths were then divided by that rate of dNTP addition to get an estimate of the time at with the inflection took place. Based on these calculations, time predictions of 11, 21.9 and 44.5 minutes with a standard deviation or step-response time of 1, 1.9 and 1.4 minutes were determined.

The number of extended strands utilized for time of step change prediction was reduced in silico. It was possible to make time of Co²⁺ addition predictions with a maximum step-response time of approximately 2.5 minutes with even just 1% of the total data analyzed. This implies that about 3000 strands of initiator DNA can give good signal recording with low step-response times. At 300 reads per sample, step-response time predictions were not able to be made.

Thus, it was determined that the smallest step-change the system can record on to DNA under these conditions is 10 minutes, with a step-response time of 1 minute. Based on these parameters, experimental set-ups for recording multiple step-changes on the same DNA strands can be designed.

Multiple Fluctuations in Co²⁺ Concentration with a Minute Resolution Recorded on to DNA:

In some embodiments, e.g., in an in vivo environment, several cationic bio-signals may be recorded in in one experimental setting. In preferred embodiments, multiple step-changes are recorded on the same DNA strand (e.g., on a single strand, or on the same plurality of strands produced in parallel in the same reaction environment).

### EXPERIMENTAL

### Example 1

### Materials and Methods

### Enzymes and Starting DNA Substrate:

Terminal deoxynucleotidyl transferase polymerase, T4 RNA ligase I, Phusion High-Fidelity PCR Master Mix with HF Buffer were purchased through New England Biolabs. Primer sequence use for extension reactions corresponded to Common Sequence I used in Illumina next generation sequencing. Primer was obtained from IDT, with standard desalting. dNTPs were obtained from Bioline.

### Extension Reaction:

Initiating primer (CS1: 5′ACACTGACGACATGGTTCTACA3′) was diluted at 0.1 μM in 1× reaction buffer along with 10 units of TdT and plus or minus 0.25 mM cobalt chloride. The reaction was started by adding 0.1 mM dNTPs in the end for a total reaction volume of 50 μL and run for 1 hour at 37 C in a Bio-Rad PCR block. Reaction was stopped by freezing at −20 or boiling at 70 C for 10 minutes. For initial testing 2 μL of the reaction was mixed with 12 μL of TBE-Urea loading dye and boiled for 10 minutes at 100 C. All of the diluted extension reaction was then loaded onto 30 μL wells 10 well 10% TBE-Urea Gel (Bio-Rad) and run for 40 minutes at 200 V. Immediately after the run was over, the gel was stained with Sybr Gold for 15 minutes and imaged on ImageQuant BioRad.

### Illumina Library Preparation and Sequencing:

Sample preparation pipeline for NGS was adapted from a previous protocol. After the extension reaction, 2 μL of the product was utilized for a ligation reaction. 22 bp universal tag, common sequence 2 (CS2) of the Fluidigm Access Array Barcode Library for Illumina Sequencers (Fluidigm), synthesized as ssDNA with a 5 phosphate modification, and PAGE purified (Integrated DNA Technologies), was blunt-end ligated to the 3′ end of extended products. Ligation reactions were carried out in 20 μL volumes and consisted of 2 μL of extension reaction, 1 μM CS1 single stranded DNA, 1× T4 RNA Ligase Reaction Buffer (New England Biolabs), and 10 units of T4 RNA Ligase 1 (New England Biolabs). Ligation reactions were incubated at 25° C. for 16 h. Ligated products were stored at −20° C. until PCR that was carried out on the same day. Ligation products were never stored at −20° C. for more than 24 hours.

PCR was performed with barcoded primer sets from the Access Array Barcode Library for Illumina Sequencers (Fluidigm) to label extension products from up to 96 individual reactions. Each PCR primer set contained a unique barcode in the reverse primer. From 5-3′ the forward PCR primer (PE1 CS1) contained a 25-base paired-end Illumina adapter 1 sequence followed by CS1. The binding target of the forward PCR primer was the reverse complement of the CS1 tag that was used as the starting DNA substrate. From 5-3′ the reverse PCR primer (PE2 BC CS2) consisted of a 24-base paired-end Illumina adapter 2 sequence, a 10-base Fluidigm barcode, and the reverse complement of CS2. CS2 DNA that had been ligated onto the 3′ end of extended products served as the reverse PCR primer-binding site. Each PCR reaction consisted of 2 μL of ligation product, 1× Phusion High-Fidelity PCR Master Mix with HF Buffer (New England Biolabs), and 400 nM forward and reverse Fluidigm PCR primers in a 20 μL reaction volume. Products were initially denatured for 30 s at 98-C, followed by 20 cycles of 10 s at 98° C. (denaturation), 30 s at 60° C. (annealing) and 30 s at 72° C. (extension). Final extensions were performed at 72° C. for 10 min. Amplified products were stored at −20° C. until clean up and pooling. Individual PCR reactions were analyzed using a 2200 TapeStation (Agilent) to determine size and quality and estimate the concentrations. Then they were pooled accordingly. Sequencing was performed using a on a MiniSeq Benchtop Sequencer (Illumina). A 15% phiX DNA control was spiked in alongside product libraries during sequencing. Fluidigm sequencing primers, targeting the CS1 and CS2 linker regions, were used to initiate sequencing. De-multiplexing of reads was performed on the instrument based on Fluidigm barcodes. Library concentration, quality analysis, and quantification were performed at the DNA services (DNAS) facility, Research Resources Center (RRC), University of Illinois at Chicago (UIC). Sequencing was performed at the W. M. Keck Center for Comparative and Functional Genomics at the University of Illinois at Urbana-Champaign (UIUC).

### Initiator Immobilization on Carboxyl Beads:

The initiator oligo (5AmMC12/TTTTTTTTT/ideoxyU/ACACTGACGACATGGTTCTACA) was immobilized on 5.28 micron carboxyl polystyrene beads (Spherotech CP-50-10) using carbodiimide conjugation. To do so, 5 mg beads were washed twice in 100 mM MES buffer pH=5.2 and resuspended in 100 μl of the same buffer. The oligo, 5AmMC12/TTTTTTTTT/ideoxyU/ACACTGACGACATGGTTCTACA, was resuspended at 100 μM in water. A 1.25M batch of EDC was prepared by dissolving 120 mg EDC (Sigma E1769, from −20 C storage) in 500 μl of 100 mM MES pH=5.2. 40 μl of the 1.25M EDC batch was mixed with 30 μl (3 nmole) of the 5Am12-fSBS3-acgtactgag oligo and 30 μl of 100 mM MES pH=5.2 and added to the beads and mixed by vortexing for 10 seconds. The suspension was rotated at room temperature overnight. After incubation overnight, the beads were washed three times with 1 mL buffer containing 250 mM Tris pH 8 and 0.01% Tween 20, each time rotating at RT for 30 min. The beads were then resuspended in 500 μl Tris-EDTA buffer with 0.01% Tween 20 and stored at 4° C. until use.

### NGS Data Processing:

For each sample, the NGS reads were first trimmed and filtered using cutadapt. Only NGS reads with both adapters, a CS1, and aCS2 sequence were kept. These parts were then trimmed off each sequence. Cutadapt parameters were a maximum error rate of 0.2, a minimum overlap of 2, a minimum length of 1, and a quality cutoff of 20. To eliminate any potential PCR bias, only reads longer than 20 nucleotides were kept using PRINSEQ, and these were then deduplicated using PRINSEQ, resulting in a set of unique reads longer than 20 nucleotides. This eliminates any possible PCR bias because it can be assumed that any duplicates longer than 20 nucleotides would arise by chance less than

$$\frac{{n}_{r e a d s}}{{4}^{20}} times ,$$

and because n_{reads} is only on the order of 10⁶, it is extremely likely that any duplicates are due to PCR bias rather than synthesized by chance (4²⁰˜10¹²). FastQC was used to quickly inspect fastq files throughout the process.
NGS Data Analysis—Effect of Primer Sequence on Base Preference: Next, for each sample the total number of A, C, G, and T nucleotides were counted across all reads using a python script. Also, in order to investigate the effects of previously added bases on the next base added, DNAp_basecount_one_file.py calculated the total number of A, C, G, and T nucleotides after a given primer sequence for all possible primer sequences of length 1 to 4 bases. For example, the total number of T nucleotides added after . . . ACCG was calculated to see if having . . . ACCG as the primer sequence affects preference for T addition.

All further analysis was done using Jupyter and the python data science stack (numpy, scipy, pandas, matplotlib, seaborn). In addition, all counts were normalized to percents (probabilities) by dividing by the total number of nucleotides. For example:

$$P ( A ) = \frac{{n}_{A}}{{n}_{A} + {n}_{C} + {n}_{G} + {n}_{T}}$$

Besides differences in overall preference of A, C, G, and T addition upon a cation change, the effects of cation addition was investigated along with the primer sequence on preference of A, C, G, and T (considered effects of up to previous 4 bases). The four previous bases of the primer sequence were assigned labels N₁, N₂, N₃, and N₄. The added base was assigned N₅. First, for each Nₛ the overall probability of its addition P(N₅) was compared with its probability of addition directly after a given nucleotide P(N₅|N₄). This was accomplished by comparing the two respective probabilities using a ratio:

$$Effec {t}_{{N}_{4}} = \frac{P ( {N}_{5} \vert {N}_{4} )}{P ( {N}_{5} )}$$

This probability ratio equals 1 if the probabilities are equal, indicating that N₄ has no effect on preference. The dependent probability P(N₅|N₄) was calculated using count data. For example:

$$P ( A \vert C ) = \frac{{n}_{C A}}{{n}_{C A} + {n}_{C C} + {n}_{C G} + {n}_{C T}}$$

which was then extended this analysis to longer primer sequences (up to 4 bases long), going back one base at a time, to determine if that base has an effect on preference:

$${Effect}_{{N}_{3}} = \frac{P ( {N}_{5} \vert {N}_{3} {N}_{4} )}{P ( {N}_{5} \vert {N}_{4} )}$$

$$Effec {t}_{{N}_{2}} = \frac{P ( {N}_{5} \vert {N}_{2} {N}_{3} {N}_{4} )}{P ( {N}_{5} \vert {N}_{3} {N}_{4} )}$$

$$Effec {t}_{{N}_{1}} = \frac{P ( {N}_{5} \vert {N}_{1} {N}_{2} {N}_{3} {N}_{4} )}{P ( {N}_{5} \vert {N}_{2} {N}_{3} {N}_{4} )}$$

The log₁₀ of each probability ratio was taken, such that values near 0 are interpreted as no effect, whereas values far from 0 indicate that a given nucleotide in the primer sequence (e.g. N₄) has an effect on the preference of N₅, the base being added.

For each base in all possible 4-base primer sequences, a two-sided T-test (scipy.stats.ttest_ind) was then applied to test the null hypothesis that the probability ratio for that base and primer sequence does not change upon addition of a given cation (either Ca or Co). This test was also applied to the overall probabilities of A, C, G, and T addition between cation conditions.

### NGS Data Analysis—Timepoint Analysis:

The data were preprocessed as described above in NGS Data Preprocessing. After that, the total numbers of A, C, G, and T across all reads were counted at each base position and normalized by the total number of nucleotides at that position, resulting in average percent A, C, G, and T at each position. Each of these values was then subtracted by the average value at the control condition (e.g. Mg only). This resulted in % difference in A, C, G, and T preference at each base position between any given sample and the control. To combine the information from all four nucleotides, the norm of the absolute values of these % differences were taken. For example, for a given position

Diffₙₒᵣₘ=\sqrt{Diff_{A}²+Diff_{C}²+Diff_{G}²+Diff_{T}²}

where

Diff_{N}=|P(N)ᵢ−P(N)₀|

At condition i, where i=0 for the control.

This overall norm percent difference was then plotted for every base across all conditions. To calculate the time at which the cation was switched, the base at which the overall norm percent difference reached half the average of the Co control norm percent difference was first calculated. Do reduce error due to rounding up or down to a specific base number, linear interpolation was used to more precisely calculate the overall point at which, along the DNA strand, the switch occurred. To calculate time, this “switch base” value was divided by the average rate of nucleotide addition (calculated from the total number of bases added across all reads and the experiment time).

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art, e.g., in biophysics, synthetic biology, bioengineering, molecular biology, biochemistry, medical science, or related fields are intended to be within the scope of the following claims.

### Example 2

Enzymes and ssDNA Substrate:

Terminal deoxynucleotidyl polymerase, T4 RNA ligase I, Phusion High-Fidelity PCR Master Mix with HF Buffer were purchased through New England Biolabs (NEB). ssDNA substrates used for extension reactions were ordered from Integrated DNA Technologies (IDT) with standard desalting. dNTPs were obtained from Bioline.

### Extension Reaction Set-Up for Reactions Analyzed by Next Generation Sequencing (NGS)

Extension Reaction for Calculating Affect of Co²⁺, Ca²⁺, Zn²⁺, and Temperature on Overall dNTP Preference of TdT:

Each extension reaction consisted of a final concentration of 10 μM ssDNA substrate (CS1: 5′ACACTGACGACATGGTTCTACA3′), 1 mM dNTP mix (each dNTP at 1 mM final concentration), 1.4×NEB TdT reaction buffer, and 10 units of TdT to a final volume of 50 μL. When testing the effect of cations, CoCl₂ was added at a final concentration of 0.25 mM, CaCl₂) at 2 mM, or Zn(Ac)₂ at 20 μM. It is important to note that reaction initiation was done by adding TdT to the ssDNA substrate mix (ssDNA substrate mix consisted of the ssDNA substrate, dNTPs and the cation). Prior to reaction initiation, the ssDNA substrate mix and TdT were stored in separate PCR strip tubes at 0° C. (on ice). The reaction was run for 1 hour at 37° C. in a Bio-Rad PCR block. When testing the effect of temperature, the same reaction mix was run on a Bio-Rad PCR block set at tested temperatures for 1 hour. Reaction was stopped by freezing at −20° C. For initial testing, 2 μL of the reaction was mixed with 12 μL of TBE-Urea (Bio-Rad) loading dye and boiled for 10 minutes at 100° C. All of the diluted extension reaction was then loaded onto 30 μL, 10 well 10% TBE-Urea Gel (Bio-Rad) and run for 40 minutes at 200 V. Immediately after the run was over, the gel was stained with Sybr Gold for 15 minutes and imaged on an ImageQuant BioRad.

### Extension Reactions for 0→1 Set-Up:

Mg²⁺ only for 1 hour (signal 0) and Mg²⁺+Co²⁺ for 1 hour (signal 1) were set up as regular extension reaction mentioned above. The 0→1 reactions where the signal changed from 0 to 1 at various times during the 1 hour extension were run starting at a total volume of 45 μL with Mg²⁺ only. 5 μL 2.5 mM CoCl₂ was added at the time the signal to change from 0 to 1 was desired. Reactions were all run for a total of 1 hour in triplicates. Fresh signal 0 and signal 1 controls were run with each set-up.

### Extension Reactions for 0→1→0 Set-Up:

Mg²⁺ only for 1 hour (signal 0) and Mg²⁺+Co²⁺ for 1 hour (signal 1) were set up as regular extension reaction mentioned above. The 0→1→0 reactions where the signal changed from 0 to 1 at 20 minutes and back to 0 at 40 minutes were run starting at a total volume of 45 μL with Mg²⁺ only. 5 μL 2.5 mM CoCl₂ was added at the time the signal to change from 0→1 was desired. For changing the signal from 1→0, since the ssDNA was suspended in reaction buffer for these set-ups, a ssDNA clean up kit (methods mentioned below) was used to remove the reaction buffer, TdT, cation and dNTPs from each reaction. All of the ssDNA collected from the ssDNA clean up kit (20 μL) was then prepared for the last part of the extension reaction. Collected ssDNA was mixed with a dNTP mix at a final concentration of 1 mM (each dNTP at 1 mM final concentration), 1.4× TdT reaction buffer and 10 units of TdT to a final volume of 50 μL. All reactions were always initiated by adding TdT in the end. Signal 0 and signal 1 controls were run for 1 hour for each set-up in triplicates and also put through the ssDNA wash step at 40 minutes. Six replicates were run for 0→1→0 reactions.

ssDNA Wash for Replacing Buffers for 0→1→0 Reactions:

For changing cation concentration from 1 to 0 the ssDNA clean-up kit (ssDNA/RNA clean/concentrator D7010) from Zymo Research was used such that all the extended ssDNA synthesized in the initial part of the experiment was retained on the column and the TdT, reaction buffer, cation and dNTPs were washed away. Each 50 μL extension reaction was individually loaded into a separate column. Protocol was followed as mentioned in the kit. ssDNA was eluted into 20 μL ddH₂O. Initial tests indicated that after using the ssDNA clean-up kit, there was little to no TdT-based extension in some replicates (data not included). This may be due some ethanol getting carried forward into the eluted ssDNA. Thus the dry spin time was extended. Two other ways to evaporate any remaining ethanol after the column dry spin step were also utilized. Either the columns were kept open in a biohood for 15 minutes to allow for evaporation, or after elution of ssDNA the 1.5 mL eppendorf tubes containing the eluted ssDNA were kept open at 45° C. for 3 minutes. Both methods gave better ethanol removal than just dry spin, and they were tried in triplicates and averaged and plotted for the time prediction analysis (FIG. 16C).

### Illumina Library Preparation and Sequencing:

The sample preparation pipeline for NGS was adapted from a previous protocol. After extension reaction, 2 μL of the product was utilized for a ligation reaction. 22 bp universal tag, common sequence 2 (CS2) of the Fluidigm Access Array Barcode Library for Illumina Sequencers (Fluidigm), synthesized as ssDNA with a 5′ phosphate modification and PAGE purified (Integrated DNA Technologies), was blunt-end ligated to the 3′ end of extended products using T4 RNA ligase. Ligation reactions were carried out in 20 μL volumes and consisted of 2 μL of extension reaction, 1 uM CS1 ssDNA, 1× T4 RNA Ligase Reaction Buffer (NEB), and 10 units of T4 RNA Ligase 1 (NEB). Ligation reactions were incubated at 25° C. for 16 hours. Ligated products were stored at −20° C. until PCR that was carried out on the same day. Ligation products were never stored at −20° C. for more than 24 hours.

PCR was performed with barcoded primer sets from the Access Array Barcode Library for Illumina Sequencers (Fluidigm) to label extension products from up to 96 individual reactions. Each PCR primer set contained a unique barcode in the reverse primer. From 5′-3′ the forward PCR primer (PE1 CS1) contained a 25-base paired-end Illumina adapter 1 sequence followed by CS1. The binding target of the forward PCR primer was the reverse complement of the CS1 tag that was used as the starting DNA substrate. From 5′-3′ the reverse PCR primer (PE2 BC CS2) consisted of a 24-base paired-end Illumina adapter 2 sequence (PE2), a 10-base Fluidigm barcode (BC), and the reverse complement of CS2. CS2 DNA that had been ligated onto the 3′ end of extended products served as the reverse PCR primer-binding site. Each PCR reaction consisted of 2 μL of ligation product, 1× Phusion High-Fidelity PCR Master Mix with HF Buffer (NEB), and 400 nM forward and reverse Fluidigm PCR primers in a 20 μL reaction volume. Products were initially denatured for 30 s at 98° C., followed by 20 cycles of 10 s at 98° C. (denaturation), 30 s at 60° C. (annealing), and 30 s at 72° C. (extension). Final extensions were performed at 72° C. for 10 min. Amplified products were stored at −20° C. until clean up and pooling. QC for individual sequencing libraries was performed as follows. 2 μL of each library was pooled into a QC pool and the size and approximate concentration was determined using Agilent 4200 Tapestation. Pool concentration was further determined using Qubit and qPCR methods. Sequencing was performed on an Illumina MiniSeq Mid Output flow cell and sequencing was initiated using custom sequencing primers targeting the CS1 and CS2 conserved sites in the library linkers. Additionally phiX control library was spiked into the run at 15-20% to increase diversity of the library clustering across the flow cell. After demultiplexing, the percent seen for each sample was used to calculate a new volume to pool for a final sequencing run with evenly balanced indexing across all samples. This pool was sequenced with metrics identical to the QC pool. Library preparation and sequencing were performed at the University of Illinois at Chicago Sequencing Core (UICSQC).

### NGS Data Preprocessing:

For each sample, the NGS reads were first trimmed and filtered using cutadapt (v1.16). Only NGS read pairs with both Illumina Common Sequence adapters, CS1 and CS2, were kept. Of these, CS2 was trimmed off each R1 sequence and CS1 was trimmed off each R2 sequence. Cutadapt parameters were set as following: a minimum quality cutoff (-q) of 30, a maximum error rate (-e) of 0.05, a minimum overlap (-O) of 10, and a minimum extension length (-m) of 1. The minimum overlap was set to be higher than the default value of 3 because extended sequences in this case are random, and it was undesirable to filter out sequences where the final 1-10 bases just happen to look like the first 10 bases of CS2 (the read must still contain a full CS2 sequence for it to be kept and subsequently trimmed, however). The 3′ (-a) adapter trimmed from the R1 reads was 5′AGACCAAGTCTCTGCTACCGTA3′ (CS2 reverse complement), and the 5′ (-A) adapter trimmed from the R2 reads was 5′TGTAGAACCATGTCGTCAGTGT3′ (CS1 reverse complement). FastQC was used to quickly inspect the output trimmed .fastq files before downstream analysis. See filter_and_trim_TdT.sh at github.com/tyo-nu/turtles for an example preprocessing script. All runs were trimmed using this script. All initial preprocessing was done on Quest, Northwestern University's high-performance computing facility, using a node running Red Hat Enterprise Linux Server release 7.5 (Maipo) with 4 cores and 4 GB of RAM, although only 1 core was used. Preprocessing took between 5 and 30 minutes depending on the number of conditions, replicates, and reads per replicate in a given run.

Finally, for each analysis, further preprocessing was performed locally. Bases that were still present in the reads but not added during the experiment were cut off. Degenerate bases (if any) that are part of the 5′ ssDNA substrate (at its 3′ end before the extension) were removed from the beginning of each sequence. Then, 5.8 bases were cut off the end of every sequence because it was determined that, on average, 5.8 bases were being added after the extension reaction during the 16 hour ligation step (FIG. 15). Because 5.8 is not an integer value, 5 bases were cut off of 80% of the sequences and 6 bases off of 20% of the sequences. Sequences with length less than 6 bases were filtered out.

### Timepoint Prediction for 0+1 Single Step Change Experiments:

All further analysis was done in python using Jupyter Notebooks. You can find all the Jupyter Notebooks used for this publication at github.com/tyo-nu/turtles. The following algorithm was applied in order to (1) read and normalize each sequence by its own length, (2) calculate a distance metric using the relative dATP, dCTP, dGTP, and dTTP percent incorporation changes between each condition and the 0 control, and (3) transform distances for all conditions into 0→1 space based on the 0 and 1 control distance values.

Each sequence was normalized by length, such that all bases in each sequence are counted across 1000 bins. For example, for a sequence of length 10, the first base would get counted in the first 100 bins, the next base in bins 100-200, and so on.

The base composition, Xᵢⱼ, was calculated in the sequence for condition, i, at each bin with position, j, using the formula for a closure (equation 1). Note that i is unique for each (condition, replicate) pair if multiple replicates are present for a given experimental condition.

$${X}_{i j} = [ \frac{{n}_{ijA}}{{\sum }_{k ∈ N} {n}_{ijk}} , \frac{{n}_{ijC}}{{\sum }_{k ∈ N} {n}_{ijk}} , \frac{{n}_{ijG}}{{\sum }_{k ∈ N} {n}_{ijk}} , \frac{{n}_{ijT}}{{\sum }_{k ∈ N} {n}_{ijk}} ] \qquad ( 1 )$$

Here, nᵢⱼₖ is the total count of dATP, dCTP, dGTP, or dTTP depending on the value of k (k∈N={A, C, G, T}) across all sequences for condition, i, at bin, j.

To calculate distance between two compositions at a given bin location (e.g. between the 0 and 1 controls at every bin), one needs to first transform the compositional data. One cannot simply take the L2 norm difference of each compositional element because the elements of a composition violate the principle of normality due to the total sum rule (all elements add up to 100%). Thus, the data is first transformed by using the center log-ratio (clr) transformation which maps this 4-component composition from a 3-dimensional space to a 4-dimensional space. One then takes the L2 norm of these transformed normal elements. This distance metric is known as the Aitchison Distance, which is used here to calculate the base composition distance, dⱼ(0,i), from the 0 control to each condition, i, at each bin, j (equation 2).

$${d}_{j} ( 0 , i ) = \sqrt{{\sum }_{K ∈ N} [ ln ( \frac{{X}_{ijk}}{g ( {X}_{ij} )} ) - ln ( \frac{{X}_{0 jk}}{g ( {X}_{0 j} )} ) ]} \qquad ( 2 )$$

N={A, C, G, T} and g(Xᵢⱼ) is the geometric mean for condition, i, and bin, j, across all four bases in N (equation 3).

$$g ( {X}_{i j} ) = \sqrt[4]{{\prod }_{k ∈ N} {X}_{ijk}} \qquad ( 3 )$$

For condition, i, and bin j, the signal, sᵢⱼ, is calculated as

$${s}_{i j} = \frac{{d}_{j} ( 0 , i ) - {d}_{j} ( 0 , 0 )}{{d}_{j} ( 0 , 1 ) - {d}_{j} ( 0 , 0 )} = \frac{{d}_{j} ( 0 , i )}{{d}_{j} ( 0 , 1 )} \qquad ( 4 )$$

where dⱼ(0,1) is the Aitchison distance between the 0 control base composition and 1 control base composition at bin, j. dⱼ(0,0)=0 for all j. If there were multiple replicates for the 0 control, their average composition was used for X₀ⱼ (and X₀ⱼₖ) in equation 2. If there were multiple replicates for the 1 control, their average composition was similarly used to calculate dⱼ(0,1) in equation 4.

Next, the switch times were estimated for each condition, i, which contains a change in signal, sᵢⱼ, (e.g. via addition of Co halfway through the reaction). For experiments with more than one change (e.g. 0→1→0), a more sophisticated approach was used and is detailed below. However, the following simpler, more intuitive approach was used to predict switch times for 0→1 and 1→0.

Switch times were estimated for a given condition, i, by (1) finding jᵢ*, the average location across all the sequences (bin position, j) at which half the 1 control signal is reached (i.e. sᵢⱼ=0.5), (2) calculating a, the ratio of the average rate of nucleotide addition for the 0 and 1 controls, and (3) using jᵢ* and a to calculate the switch time, tᵢ* using equations 5 and 6. For a derivation of equation 5, see supplementary methods.

$$\begin{matrix}{t}_{i}^{*} = \frac{α {t}_{expt}}{\frac{1}{{j}_{i}^{*}} + α - 1} & ( 5 ) \\ where & \\ α = \frac{\overline{{r}_{a , ctrl}}}{\overline{{r}_{b , ctrl}}} & ( 6 )\end{matrix}$$

r̅_̅{̅a̅,̅c̅t̅r̅l̅}̅ is the average synthesis rate of the first environmental condition before the switch. For example, r̅_̅{̅a̅,̅c̅t̅r̅l̅}̅ would be calculated using the 0 control for the condition, 0→1, but the 1 control for the condition, 1→0. The average synthesis rate is calculated by dividing the average extension length by the duration of the experiment. r̅_̅{̅b̅,̅c̅t̅r̅l̅}̅ is the average synthesis rate for the second environmental condition (after the switch).

### Timepoint Prediction for 0→1→0 Multiple Fluctuations Experiment:

To predict the Co²⁺ condition in the 0→1→0 experiment, the algorithm discussed herein was used for decoding continuous concentrations. The input to this algorithm is the amount of signal on every nucleotide. Here, the signal is sᵢⱼ from the previous section. The algorithm uses this information to predict continuous values of Co²⁺ between 0 and 1 for all time points that are most likely to produce the amount of signal on the nucleotides. To binarize these predictions, a threshold of 0.5 was set. To be able to predict the values of Co²⁺, the algorithm requires knowledge of the expected amount of signal in the 0 and 1 control conditions. Here, this is the average signal across nucleotides in the 0 or 1 control experiments. The algorithm also requires knowledge of the rate of nucleotide addition. Here, an inverse Gaussian distribution was fit to the average experimental dNTP addition rate distribution (the distribution of the sequence lengths divided by the experiment time) from the control experiments. Note that this algorithm also assumes that the rate of dNTP addition is independent of the cation concentration. Thus, when making predictions in the 0→1→0 experiment, the disclosed data do not account for differences in the rate of dNTP addition distributions between the 0 and 1 conditions. A future algorithm that takes this difference into account could yield more accurate predictions.

In Silico Simulations of Experiments with More than 3 Bits:

Using the average dNTP addition rate from experiments, and the amount of signal in the control conditions, additional experiments with 1,000 strands were simulated. Each simulated experiment had at least 3 bits (pulses of being in either the 1 or 0 condition), where each bit was randomly chosen to be 0 or 1. All nucleotides that were added during the 0 or 1 condition had the signal associated with these control conditions. More specifically, to account for the experimental variability in signals within a given control condition, nucleotide signals were sampled from a Normal distribution determined by the experimental variability of nucleotide signals within the control conditions. Using the signal of the simulated nucleotides, the algorithm disclosed herein was used for decoding binary concentrations. The accuracy is the percentage of bits correctly classified as 0 or 1.

Extension Reaction with Individual dNTPs for Testing Effect of Co²⁺:

For initial testing to show Co²⁺ dependent dNTP preference change the ssDNA substrate used was AMD006: 5′AGGCTAGTCGTCTGTATAGG3′. Total reaction volume was 25 μL with 0.1 μM ssDNA substrate, 1×NEB TdT reaction buffer, and 0.1 mM of each dNTP tested. Final concentration of CoCl₂ in the test reaction was 0.25 mM. Reactions were initiated by addition of 5 units of TdT per reaction. Reactions were run for 30 minutes at 37° C. and stopped by boiling at 70° C. for 10 minutes. Then, 8 μL of the reaction was mixed with 12 μL of TBE-Urea loading dye and boiled for 10 minutes at 100° C. All of the diluted extension reaction was then loaded onto 30 μL, 10 well 10% TBE-Urea Gel (Bio-Rad) and run for 40 minutes at 200 V. Immediately after the run was over, the gel was stained with Sybr Gold for 15 minutes and imaged on ImageQuant BioRad.

### Extension Reactions for 1→0 Set-Up:

Mg²⁺ only for 1 hour (signal 0) and Mg²⁺+Co²⁺ for 1 hour (signal 1) were set-up as regular extension reactions mentioned in Materials and Methods. The 1→0 reactions where the signal changed from 1 to 0 at 40 minutes were put through a ssDNA was step at 40 minutes. ssDNA wash to remove cations, TdT and dNTPs was done exactly as mentioned in Materials and Methods. Reactions were all run for 1 hour in triplicates. Signal 0 and signal 1 controls were run for 1 hour for each set-up in triplicates and also put through the ssDNA wash step at 40 minutes.

### Derivation of Equation 5

The derivation of Equation 5 was started by deriving the equations for the average rate before the switch (r_{A}) and after the switch (r_{B}) for condition, i:

$$\begin{matrix}{r}_{a , i} = \frac{{j}_{i}^{*}}{{t}_{i}^{*}} & ( 1 a ) \\ {r}_{b , i} = \frac{1 - {j}_{i}^{*}}{{t}_{expt} - {t}_{i}^{*}} & ( 2 a )\end{matrix}$$

where jᵢ* is the average location in the sequences (length fraction, 0 to 1) at which the signal, sᵢⱼ, reaches 0.5 (Equation 4), tᵢ* is the switch time, and tₑₓₚₜ is the total duration of the experiment. Because r_{a,i} and r_{b,i} can be estimated from average rates of the 0 and 1 controls across replicates (r̅_̅{̅a̅,̅c̅t̅r̅l̅}̅ and r̅_̅{̅b̅,̅c̅t̅r̅l̅}̅), their ratio can be used to combine equation 1a and 2a, above to write

$$\frac{\overline{{r}_{a , ctrl}}}{\overline{{r}_{b , ctrl}}} \approx \frac{{r}_{a , i}}{{r}_{b , i}} = \frac{{j}_{i}^{*}}{{t}_{i}^{*}} ( \frac{{t}_{expt} - {t}_{i}^{*}}{1 - {j}_{i}^{*}} ) \qquad ( 3 a )$$

Solving for tᵢ* to get equation 5:

$$\begin{matrix}{t}_{i}^{*} = \frac{α {t}_{expt}}{\frac{1}{{j}_{i}^{*}} + α - 1} & ( 5 ) \\ where & \\ α = \frac{\overline{{r}_{a , ctrl}}}{\overline{{r}_{b , ctrl}}} & ( 4 a )\end{matrix}$$

Equation 5 was used for time prediction (tᵢ*) after calculating jᵢ* for a given condition and a from the 0 and 1 controls. In equation 4a, a is the first condition before the switch (0 or 1) and b is the condition after the switch (1 or 0).
Extensions Reaction Set-Up for Calculating Rate of dNTP Addition:

Each extension reaction consisted of a final concentration of 10 μM initiating ssDNA substrate, 1 mM dNTP mix (each dNTP at 1 mM final concentration), 1.4×NEB TdT reaction buffer, and 10 units of TdT to a final volume of 50 μL. The ssDNA substrate used for this extension reaction was CS1_5N: 5′ACACTGACGACATGGTTCTACA(N1:25154515)(N1)(N1)(N1)(N1)3′. It has been shown (data not included) that the identity of the last 5 bases on the 3′ end of the substrate affects the identity of the dNTP added to the ssDNA substrate. Thus, a ssDNA substrate (CSL_5N) was purchased with the last 5 bases having the base composition same as TdT dNTP preference under signal 0 (25% dATP, 15% dCTP, 45% dGTP and 15% dTTP). This primer was used for this set-up, but it was not believed that the identity of the primer affect the rate of dNTP addition. The reactions were initiated upon addition of TdT and run at 37° C. for 2 hours. 2 μL of sample was collected and immediately frozen (on ice, 0° C.) at 30 s, 1 min, 2 min, 3 min, 4 min, 5 min, 10 min, 20 min, 30 min, 45 min, 60 min, 92 min and 120 min. Subsequently, each sample was put through the ligation and Illumina library generation process as mentioned in Materials and Methods.

Test Set-Up for Checking ssDNA Clean-Up Kit Bias:

Mg²⁺ only for 1 hour (signal 0) and Mg²⁺+Co²⁺ for 1 hour (signal 1) were set up as regular extension reactions mentioned in Materials and Methods. The 0→1 reactions where the signal changed from 0 to 1 during the 1 hour extension were run starting with 45 μL with Mg²⁺ only. 5 μL of 2.5 mM CoCl₂ was added at 10 min. Reactions were all run for 1 hour in triplicates. Fresh signal 0 and signal 1 controls were run for 1 hour with each set-up. 2 μL of extension reaction was used for ligation (“No Wash” set of samples). Ligation and subsequent PCR steps for Illumina library generation were followed as mentioned in Materials and Methods. Rest of the 48 uL of extension reaction was washed using the ssDNA clean-up kit. Protocol was followed as mentioned in the kit. ssDNA was eluted into 25 μL of ddH₂O and 2 μL of that was used for ligation (“Wash” set of samples). Ligation and subsequent PCR steps for Illumina library generation were followed as mentioned in Materials and Methods. Data obtained from Illumina sequencing was analyzed for the “No Wash” and “Wash” set of samples. Further, switch time calculations were carried out as mentioned previously (FIG. 14).

### REFERENCES

The following references are herein incorporated by reference in their entireties for all purposes.
1. Antebi, Y. E., Nandagopal, N. & Elowitz, M. B. An operational view of intercellular signaling pathways. Curr. Opin. Syst. Biol. 1, 16-24 (2017).
2. Sheth, R. U. & Wang, H. H. DNA-based memory devices for recording cellular events. Nat. Rev. Genet. 19, 718-732 (2018).
3. Purvis, J. E. & Lahav, G. Encoding and decoding cellular information through signaling dynamics. Cell 152, 945-56 (2013).
4. Church, G. M., Gao, Y. & Kosuri, S. Next-Generation Digital Information Storage in DNA. Science (80-. ). 337, 1628-1628 (2012).
5. Goldman, N. et al. Towards practical, high-capacity, low-maintenance information storage in synthesized DNA. Nature 494, 77-80 (2013).
6. Erlich, Y. & Zielinski, D. DNA Fountain enables a robust and efficient storage architecture. Science (80-. ). 355, 950-954 (2017).
7. Kording, K. P. Of toasters and molecular ticker tapes. PLoS Comput. Biol. 7, 1-5 (2011).
8. Grass, R. N., Heckel, R., Puddu, M., Paunescu, D. & Stark, W. J. Robust Chemical Preservation of Digital Information on DNA in Silica with Error-Correcting Codes. Angew. Chemie Int. Ed. 54, 2552-2555 (2015).
9. Shendure, J. et al. DNA sequencing at 40: past, present and future. Nature 550, 345-353 (2017).
10. Weinberg, B. H. et al. Large-scale design of robust genetic circuits with multiple inputs and outputs for mammalian cells. Nat. Biotechnol. 35, 453-462 (2017).
11. Chiu, T.-Y. & Jiang, J.-H. R. Logic Synthesis of Recombinase-Based Genetic Circuits. Sci. Rep. 7, 12873 (2017).
12. Perli, S. D., Cui, C. H. & Lu, T. K. Continuous genetic recording with self-targeting CRISPR-Cas in human cells. Science (80-. ). 353, aag0511-aag0511 (2016).
13. Farzadfard, F. & Lu, T. K. Genomically encoded analog memory with precise in vivo DNA writing in living cell populations. Science (80-. ). 346, 1256272-1256272 (2014).
14. Tang, W. & Liu, D. R. Rewritable multi-event analog recording in bacterial and mammalian cells. Science (80-. ). 360, eaap8992 (2018).
15. McKenna, A. et al. Whole-organism lineage tracing by combinatorial and cumulative genome editing. Science (80-. ). 353, aaf7907 (2016).
16. Frieda, K. L. et al. Synthetic recording and in situ readout of lineage information in single cells. Nature 541, 107-111 (2017).
17. Sheth, R. U., Yim, S. S., Wu, F. L. & Wang, H. H. Multiplex recording of cellular events over time on CRISPR biological tape. Science 358, 1457-1461 (2017).
18. Shipman, S. L., Nivala, J., Macklis, J. D. & Church, G. M. Molecular recordings by directed CRISPR spacer acquisition. Science 353, aaf1 175 (2016).
19. Shipman, S. L., Nivala, J., Macklis, J. D. & Church, G. M. CRISPR-Cas encoding of a digital movie into the genomes of a population of living bacteria. Nature (2017). doi:10.1038/nature23017
20. Kalhor, R., Mali, P. & Church, G. M. Rapidly evolving homing CRISPR barcodes. Nat. Methods 14, 195-200 (2017).
21. Stosiek, C., Garaschuk, O., Holthoff, K. & Konnerth, A. In vivo two-photon calcium imaging of neuronal networks. at <www.pnas.orgcgidoi10.1073pnas.1232232100>
22. Ziv, Y. et al. Long-term dynamics of CAl hippocampal place codes. Nat. Neurosci. 16, 264-6 (2013).
23. Fosque, B. F. et al. Labeling of active neural circuits in vivo with designed calcium integrators. Science (80-. ). 347, 755-760 (2015).
24. Zamft, B. M. et al. Measuring cation dependent DNA polymerase fidelity landscapes by deep sequencing. PLoS One 7, (2012).
25. Marblestone, A. H. et al. Conneconomics: The Economics of Large-Scale Neural Connectomics. bioRxiv 001214 (2013). doi:10.1101/001214
26. Marblestone, A. H. et al. Physical principles for scalable neural recording. Front. Comput. Neurosci. 7, 1-34 (2013).
27. Marblestone, A. H. et al. Rosetta Brains: A Strategy for Molecularly-Annotated Connectomics. (2014). at <http://arxiv.org/abs/1404.5103>
28. Glaser, J. I. et al. Statistical Analysis of Molecular Signal Recording. PLoS Comput. Biol. 9, (2013).
29. Farzadfard, F. & Lu, T. K. Emerging applications for DNA writers and molecular recorders. Science 361, 870-875 (2018).
30. Carter, K. P., Young, A. M. & Palmer, A. E. Fluorescent sensors for measuring metal ions in living systems. Chem. Rev. 114, 4564-601 (2014).
31. Dean, K. M., Qin, Y. & Palmer, A. E. Visualizing metal ions in cells: an overview of analytical techniques, approaches, and probes. Biochim. Biophys. Acta 1823, 1406-15 (2012).
32. Zador, A. et al. Probing the connectivity of neural circuits at single-neuron resolution using high-throughput DNA sequencing. Nat. Preced. (2011). doi:10.1038/npre.2011.6452.1
33. Motea, E. A. and A. J. B. Terminal Deoxynucleotidyl Transferase: The Story of a Misguided DNA Polymerase. 21, 253-260 (2015).
34. Chang, M. S. & Bollum, F. J. Multiple Roles of Divalent Deoxynucleotidyltransferase Cation in the Terminal Reaction*. 265, 17436-17440 (1990).
35. Fowler, J. D. & Suo, Z. Biochemical, Structural, and Physiological Characterization of Terminal Deoxynucleotidyl Transferase. 2092-2110 (2006). doi:10.1021/cr040445w
36. Deibel, M. R. & Coleman, M. S. Biochemical properties of purified human terminal deoxynucleotidyltransferase. J. Biol. Chem. 255, 4206-12 (1980).
37. Romain, F., Barbosa, I., Gouge, J., Rougeon, F. & Delarue, M. Conferring a template-dependent polymerase activity to terminal deoxynucleotidyltransferase by mutations in the Loop1 region. Nucleic Acids Res. (2009). doi:10.1093/nar/gkp460
38. de Paz, A. M. et al. High-resolution mapping of DNA polymerase fidelity using nucleotide imbalances and next-generation sequencing. Nucleic Acids Res. 46, e78-e78 (2018).

What is claimed:

1. A method of identifying a biological signal comprising exposing a template-independent DNA polymerase to an organic environment comprising deoxyribonucleotide triphosphates (dNTPs) and a variable, allowing the DNA polymerase to add dNTPs to a DNA substrate, and isolating the DNA substrate; wherein the dNTP content of the DNA substrate corresponds to the concentration of the variable in the organic environment.

2. The method of claim 1, wherein the template-independent DNA polymerase is a terminal deoxynucleotidyl transferase (TdT).

3. The method of claim 1 or 2, wherein the organic environment is the inside of a cell.

4. The method of claim 3, wherein the cell is a neuron.

5. The method of claim 1 or 2, wherein the organic environment is extracellular space between cells in a tissue or organ.

6. The method of any one of claims 1-5, wherein the variable is a cation.

7. The method of claim 6, wherein the cation is selected from the group consisting of Co²⁺, Ca²⁺, and Zn²⁺.

8. The method of any one of claims 1-7, wherein the DNA substrate is a single stranded DNA.

9. The method of any one of claims 1-8 further comprising sequencing the DNA substrate to determine the dNTP content of the DNA substrate.

10. The method of claim 9, wherein sequencing the DNA substrate comprises next-generation sequencing (NGS), true single molecule sequencing (tSMS), 454 sequencing, SOLiD sequencing, ion torrent sequencing, single molecule real time (SMRT) sequencing, Illumina sequencing, nanopore sequencing, or chemical-sensitive field effect transistor (chemFET) sequencing.

11. The method of any one of claims 1-10 further comprising determining the concentration of the variable based on the sequence of the DNA substrate.

12. The method of claim 11, wherein the concentration is a relative concentration over time.

13. The method of claim 11, wherein the concentration is an absolute concentration over time.

14. The method of any one of claims 11-13, wherein determining the concentration comprises (a) reading the dNTPs on one strand and using a hidden Markov model to assign the most likely cation state at each base; or (b) reading the dNTPs of many strands in parallel, where at each time point, one base from each strand is used to estimate the incorporation frequency for that time point.

15. A method of detecting a change in a variable within a cell, comprising exposing a template-independent DNA polymerase within a cell to a variable, allowing the DNA polymerase to transcribe a DNA substrate, isolating the DNA substrate, and determining whether the concentration of the variable changed over time based on the sequence of the DNA substrate; wherein the dNTP content of the DNA substrate corresponds to the amount of the variable in the cell during transcription of the DNA substrate.

16. The method of claim 15, wherein the template-independent DNA polymerase is a terminal deoxynucleotidyl transferase (TdT).

17. The method of claim 15 or 16, wherein the cell is a neuron.

18. The method of any one of claims 15-17, wherein the variable is a cation.

19. The method of claim 18, wherein the cation is selected from the group consisting of Co²⁺, Ca²⁺, and Zn²⁺.

20. The method of any one of claims 15-19, wherein the DNA substrate is a single stranded DNA.

21. The method of any one of claims 15-20 further comprising sequencing the DNA substrate to determine the dNTP content of the DNA substrate.

22. The method of claim 21, wherein sequencing the DNA substrate comprises next-generation sequencing (NGS), true single molecule sequencing (tSMS), 454 sequencing, SOLiD sequencing, ion torrent sequencing, single molecule real time (SMRT) sequencing, Illumina sequencing, nanopore sequencing, or chemical-sensitive field effect transistor (chemFET) sequencing.

23. The method of any one of claims 15-22, wherein determining whether the concentration of the variable changed over time comprises (a) reading the dNTPs on one strand and using a hidden Markov model to assign the most likely cation state at each base; or (b) reading the dNTPs of many strands in parallel, where at each time point, one base from each strand is used to estimate the incorporation frequency for that time point.

24. The method of any one of claims 15-23, wherein determining whether the concentration of the variable changed over time comprises determining the relative concentration of the variable over time.

25. The method of any one of claims 15-23, wherein determining whether the concentration of the variable changed over time comprises determining the relative concentration of the absolute over time.
